(19) United States
(12) Patent Application Publication
(10) Pub. No.: US 2026/0130983 A1
(43) Pub. Date: May 14, 2026

(54) STABILIZATION OF VIRUS-BASED THERAPEUTIC AGENT

(71) Applicant: ELAREX INC., Burlington (CA)

(72) Inventor: Jeremy Andrew IWASHKIW, Burlington (CA)

(21) Appl. No.: 19/118,699

(22) PCT Filed: Oct. 6, 2023
(86) PCT No.: PCT/CA2023/051334

§ 371 Date: Apr. 4, 2025

Related U.S. Application Data
(60) Provisional application No. 63/414,245, filed on Oct. 7, 2022.

(51) Int. Cl. A61K 39/205; A61K 9/19; A61K 39/235; A61K 47/26; A61K 47/36; A61K 47/42; C12N 7/04; C12N 15/861

(52) U.S. Cl. CPC A61K 39/205; A61K 9/19; A61K 39/235; A61K 47/26; A61K 47/36; A61K 47/42; C12N 7/04; C12N 15/861; C12N 2710/10043; C12N 2760/20243

(57) ABSTRACT

A virus-based active agent is mixed with trehalose and water and dried. The mixture may also contain one or more of pullulan and albumin. The mixture may be dried to a moisture content of 0.1-10%. The drying may be under vacuum sufficient to produce a foam. Some or all of the drying may be at a temperature in the range of 15-40° C., or at a temperature in the range of 1-15° C., or both. The active agent may be based on a vesicular stomatitis virus (VSV) or an adenovirus (AdV). The dried mixture may be stored at a temperature in the range of 1-55° C. A composition includes a virus, which may be a derived or modified form of a virus such as VSV or AdV. The composition also includes trehalose and optionally one or more of pullulan, and albumin. The composition may be used for a virus-based vaccine.

### RELATED APPLICATION

This application claims the benefit of, and priority from, U.S. Provisional Application No. 63/414,245, filed on Oct. 7, 2022, which is incorporated by reference.

### FIELD

This specification relates to a method for stabilizing a a virus-based active agent, for example a viral vector vaccine, and to a composition comprising a virus-based active agent.

### BACKGROUND

Vesicular stomatitis virus (VSV) is a negative stranded enveloped RNA virus of the Rhabdoviridae family. VSV has two known serotypes, New Jersey (VSNJV) and Indiana (VSIV), with various strains in each serotype. Recombinant VSV (rVSV) platforms have been proposed as vaccines for viral diseases in humans and have been studied, for example, for use as therapeutic cancer vaccines. rVSV platforms may have one or more genetic modifications, for example modifications to attenuate the virus or the addition of one or more antigenic inserts. rVSV platforms have mild pathogenicity in humans but can induce humoral and cellular immune responses. In one example, rVSV-EBOV, in which the glycoprotein of VSV is exchanged with Ebola glycoprotein, is effective to inhibit Ebola infection in humans at doses between 106-10⁸ PFU/dose when administered by intra-muscular injection. Unfortunately, VSV is less thermally stable than many other viruses. The rVSV-EBOV vaccine is stored in a frozen liquid formulation at −70° C. and loses effectiveness rapidly when thawed.

Adenoviruses (AdV) are non-enveloped DNA viruses with many serotypes. AdV-vectored vaccines may be derived from chimpanzee serotypes or human serotypes, for example AdV serotype 5. Recombinant AdV vectors are typically more thermally stable than other viral vectors and accordingly may be useful for vaccines having longer, or higher temperature, storage requirements.

US Patent Application Publication No. US 2019/0111006 A1 describes a method of preserving one or more biological species in a polymer matrix comprising pullulan and trehalose. The method includes combining the one or more biological species, an aqueous pullulan solution and an aqueous trehalose solution and drying the resultant mixture to provide a solid polymeric matrix. In some examples, the biological species is a live-attenuated viral vaccine and an inactivated viral vaccine.

Toniolo et al., Spray dried VSV-vectored vaccine is thermally stable and immunologically active in vivo, Scientific Reports 10, Article number: 13349 (2020), describes stabilizing a VSV-based vaccine in compositions comprising one or more of trehalose, dextran and mannitol. The compositions were spray dried. A composition comprising trehalose and another composition comprising trehalose and dextran mixed at a 3:1 ratio produced about a 4 log PFU loss after 7 days of storage of the spray dried product at 37° C.

Berg et al., Stability of Chimpanzee Adenovirus Vectored Vaccines (ChAdOx1 and ChAdOx2) in Liquid and Lyophilised Formulations, Vaccines, 2021: 9(11):1249, describes stabilizing an AdV-based vaccine in compositions including, among other things, inulin and mannitol. A freeze-dried example had an infectivity loss of 2 log after storage at 45° C. and an infectivity loss of about 1.5 log after 60 days of storage at 30° C.

### SUMMARY

This specification describes a method of preserving and/or stabilizing a virus. The virus may be an active agent of a virus-based vaccine such as a viral vector vaccine. In some examples, the virus is a recombinant virus and/or a derived from a VSV or an AdV. The virus is mixed with trehalose, a buffer and water. Optionally, the mixture may also contain pullulan, albumin or both. The mixture is dried, for example by foam drying. The mixture may be dried to a moisture content of 1 to 10%. In some examples, the drying is at two or more temperatures. The dried mixture may be stored, for example at a temperature in the range of 1-55° C. Optionally, the dried mixture may be dissolved in water, optionally in the form of an aqueous buffer, to form an injectable liquid vaccine.

This specification also describes a composition. The composition includes a virus. The virus may be an active agent of a virus-based vaccine such as a live-attenuated viral vaccine or a viral vector vaccine. In some examples, the virus is a recombinant virus and/or a derived from a VSV or an AdV. The composition also includes trehalose and a buffer. The composition may also include pullulan, albumin or both. The composition may have a moisture content of 1 to 10%. The composition may include a foamed glass incapsulating the virus. The composition may be used, for example, in a virus-based vaccine such as a viral vector vaccine.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DRAWINGS

Other features and the embodiments of the application will now be described in greater detail with reference to the attached drawings in which:

FIGS. 1A, 1B and 1C show the results of the analysis of an exemplary pullulan/trehalose (PT)/CM/BSA/NaCl composition and a comparative sorbitol and gelatin (control) composition, both dried at 20° C. including Residual Moisture (RM) (FIG. 1A), b) titer measured after storage at 37° C. for 189 days (FIG. 1B), and c) total log loss measured for 22 weeks (FIG. 1C).

FIGS. 2A to 2G show the results of the analysis of exemplary PT/CM/BSA/NaCl and Tris/Trehalose compositions dried at 4° C./25° C. including FIG. 2A: RM, FIG. 2B: total log loss measured for 32 weeks at 4° C., FIG. 2C: total log loss measured for 32 weeks at 25° C., FIG. 2D: total log loss measured for 32 weeks at 37° C., FIG. 2E: titer time course measured for 32 weeks at 4° C., FIG. 2F: titer time course measured for 32 weeks at 25° C., and FIG. 2G: titer time course measured for 32 weeks at 4° C.

FIGS. 3A-3E show the results of the analysis of exemplary PT/CM/BSA/NaCl and Tris/Trehalose compositions in FBS free media dried at 4° C./25° C. including FIG. 3A: total protein, FIG. 3B: RM, FIG. 3C: titer time course measured for 14 days at 37° C., FIG. 3D: total log loss measured for 14 days, and FIG. 3E: total log loss measured for 14 days at 37° C.

FIG. 4 shows the results of the RM analysis of exemplary PT/CM/BSA/NaCl composition in FBS free media with various amounts of BSA, pullulan and trehalose, dried at 4° C./25° C.

FIGS. 5A and 5B show the results of analysis of the exemplary PT/CM/BSA/NaCl compositions in FIG. 4 including FIG. 5A: titer time course measured for 14 days, and FIG. 5B: stability time course measured for 14 days at 37° C.

FIGS. 6A-6G show the results of the analysis of exemplary PT/CM/BSA/NaCl and Tris/Trehalose compositions dried at 4° C./25° C. including FIG. 6A: RM, FIG. 6B: titer time course measured for 20 weeks at 4° C., FIG. 6C: titer time course measured for 20 weeks at 25° C., FIG. 6D: titer time course measured for 4 weeks at 37° C., FIG. 6E: total log loss measured for 20 weeks at 4° C., FIG. 6F: total log loss measured for 20 weeks at 25° C., FIG. 6G: total log loss measured for 4 weeks at 37° C.,

FIG. 7 is a bar chart of results of the analysis of exemplary PT/CM/BSA/NaCl composition dried at 4° C./25° C. with varying P/T concentrations showing titer loss measured for 14 days.

FIG. 8 is a line graph corresponding to FIG. 7.

FIG. 9 shows the RM of exemplary PT/CM/BSA/NaCl compositions with varying treatment of viral stock and sucrose addition dried at 15° C./25° C.

FIG. 10 shows the total log loss measured for 14 days at 37° C. for the formulations in FIG. 9.

FIG. 11 shows the RM of various exemplary compositions.

FIG. 12 shows the titer of the compositions of FIG. 11 after storage at 37° C.

FIG. 13 shows the loss of titer of the compositions of FIG. 11 after storage at 37° C.

FIG. 14 shows the RM of various exemplary compositions.

FIG. 15 shows the titer of the compositions of FIG. 14 after storage at 37° C.

FIG. 16 shows the loss of titer of the compositions of FIG. 14 after storage at 37° C.

FIG. 17 shows infectivity unit (IU) loss of two AdV formulations after foam drying and storage at 37° C.

FIG. 18 shows infectivity unit (IU) loss of the two AdV formulations of FIG. 17 after foam or freeze drying and storage at 37° C.

FIG. 19 shows infectivity unit (IU) loss of three AdV formulations stored at 37° C.

FIG. 20 shows infectivity unit (IU) loss of an AdV formulation stored at three temperatures.

FIG. 21 shows infectivity unit (IU) loss of two AdV formulations stored at 55° C.

### DETAILED DESCRIPTION

### I. Definitions

Unless otherwise indicated, the definitions described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise. For example, an embodiment including “a biomolecule” should be understood to present certain aspects with one biomolecule or two or more additional biomolecules.

In embodiments comprising an “additional” or “second” component, such as an additional or second biomolecule, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “process loss” as used herein refers to a loss in titer during the process of composition formulation (i.e. buffer exchange and addition of further excipient solution) and drying. Without intended to be limited by theory, process loss appears to be primarily related to the drying procedure. The term “storage stability” as used herein refers to titer loss, if any, during storage of the dried product at one or more temperatures. The term “stability” as used herein may refer to process loss or storage stability or both. Concentrations given in % herein are w/v unless stated otherwise.

The term “method of the application” or “method of the present application” and the like as used herein refers to a method of preserving and/or stabilizing a virus.

The term “composition of the application” or “composition of the present application” and the like as used herein refers to a composition comprising a virus.

The term “preserving” or “preservation” as used herein with respect to the virus means to maintain at least a measurable or detectable level of function or activity for the virus for a desired period of time under specified conditions.

The term “stabilizing” or “stabilization” as used herein with respect to a virus refers to any reduction in the degradation or loss of activity of the virus compared to a control.

The term “pullulan” as used herein refers to a polysaccharide polymer comprising maltotriose units. Optionally, pullulan may be a natural polysaccharide which is produced by Aureobasidium pullulans. The pullulan used in examples herein has a molecular mass of 200 kDa.

The term “trehalose” as used herein refers to a disaccharide commonly used as a cryoprotectant. Trehalose may be (D)-(+)-trehalose which is a disaccharide composed of two glucose molecules bound together via the α,α-1,1-glucosidic linkage.

The term “vaccine” as used herein may mean, where appropriate given the context, an antigen of a vaccine, but does not necessarily exclude the presence of other parts of a vaccine, such as an adjuvant or diluent.

The term “essentially free from” as used herein means that the presence of the stated features, elements, or components, is in an amount that does not materially affect the characteristics of the composition or material being referenced.

As used herein, the term “effective amount” or “therapeutically effective amount” means an amount that is effective, at dosages and for periods of time necessary, to achieve a desired result.

### II. Methods of the Application

The present application includes a method of preserving and/or stabilizing a virus, the method including combining the virus with trehalose, water and a buffer, optionally with pullulan and/or albumin, to produce a composition; and drying the to produce a dried composition. The dried composition may include a glass encapsulating particles of the virus.

In some embodiments, the virus is a virus in any recombinant, derived or modified form. In some embodiments, the virus is a live-attenuated virus, an inactivated virus, a viral vector or a recombinant virus. In some embodiments, the virus is an RNA virus, optionally an enveloped RNA virus. In some embodiments, the virus is a vesicular stomatitis virus (VSV). In some embodiments, the VSV is a recombinant VSV. In some embodiments, the virus is an adenovirus or derived from an adenovirus. In some embodiments, the virus is formulated for administration in a biological preparation. In some embodiments, the virus is formulated for administration as a vaccine.

In some embodiments, samples of virus may be provided from suppliers in a buffer and may contain remnants of the virus manufacturing process. In some embodiments, the samples of virus may be purified to remove manufacturing process remnants according to any purification method known in the art. In some embodiments, a buffer exchange may be performed to substantially replace a buffer originally supplied with the virus samples with a new buffer. In some embodiments, when a virus sample or originally supplied sample buffer is used that contains any component of a composition described in the present application, the amount of the component may be adjusted to account for the amount carried over from the virus sample or the originally supplied buffer.

In some embodiments, the buffer is any buffer that maintains the pH of the composition of the application within the range of 6.8 to 8.2. In some embodiments, the buffer is a CM buffer or a tris(hydroxymethyl)aminomethane (Tris) buffer. In some embodiments, the buffer is a CM buffer. The CM buffer is prepared by mixing 2.5 g MgSO₄*7H₂O (10 mM), 0.735 g CaCl2 (10 mM), 0.05 g gelatin (0.005 mM) and 6 mL 1 M Tris-HCl (50 mM), with water for a final volume of 1 L. In some embodiments, the buffer is a Tris buffer or a Tris-HCl buffer. The Tris buffer includes 10-50 mM of Tris. In some embodiments, the buffer maintains the pH of the composition in the range of 6.8 to 8.2, in the range of 6.9 to 8.1, or in the range of 7.2-7.5. No differences in stability have been detected for compositions having pH in a range of 6.9 to 8.1. Tris and Tris-based buffers are suitable for compositions having a pH of at least 7. In some embodiments, the buffer is present in a liquid composition at a concentration of about 5 mM to about 20 mM. In some embodiments, the buffer is present in the dry composition at a concentration of about 0.5 wt % to about 10 wt %. In some embodiments, the buffer is present in the dry composition at a concentration of about 1 wt %, about 1.5 wt %, about 2 wt %, about 4 wt %, or about 8 wt % and values therebetween.

In some embodiments, other buffers, for example a Histidine buffer, may be used. In some embodiments, the buffer does not contain substantial amounts of crystal forming components. For example, phosphate-buffered saline (PBS) might reduce the performance of the composition of the application. In some embodiments, CM buffer may produce sulfate crystals and accordingly a Tris-HCl buffer or other buffer may be preferred over a CM buffer in some examples.

In some embodiments, the trehalose is present in the liquid composition at a concentration of about 1.25% (w/v) to about 15% (w/v). In some embodiments, the trehalose is present in the composition at a concentration of about 1.25% (w/v), about 2.5% (w/v), about 5% (w/v), or about 10% (w/v). Trehalose is available from a variety of commercial sources. In some embodiments, the trehalose is present in the dry composition in a concentration of about 30 wt % to about 70 wt %, or about 70 wt % to 99 wt %. In some embodiments, the trehalose is present in the dry composition in a concentration of about 35% wt %, about 35 wt %, about 40 wt %, about 45 wt %, about 50 wt %, about 55 wt %, about 60 wt %, about 65 wt %, or about 80 wt %, about 90 wt %, or about 95 wt % and values therebetween.

In some embodiments, the composition comprises pullulan. In some embodiments the pullulan has a molecular weight in the range of about 100,000 to about 200,000. Pullulan having such molecular weights is commercially available. In some embodiments, the pullulan is present in the composition in a concentration of about 0.5% to about 15%. In some embodiments, the pullulan is present in the composition at a concentration of about 0.625%, about 1.25%, about 2.5%, about 5%, or about 10%. In some embodiments, the pullulan is present in the dry composition in a concentration of about 15% wt % to about 50 wt %. In some embodiments, the pullulan is present in the dry composition in a concentration of about 20 wt %, about 25 wt %, about 25 wt %, about 30 wt %, about 35 wt %, about 40 wt %, or about 45 wt % and values therebetween.

In some embodiments, unless stated otherwise and when the composition comprises trehalose and pullulan, “PT” refers to a solution having pullulan and trehalose. Optionally, different ratios of pullulan to trehalose may be used. PT solutions may be viscous and can be prepared independently of a virus containing buffer and then added to the virus containing buffer, for example at a ratio of PT solution to virus containing buffer in the range of 1:1 to 9:1 or 4:1 to 9:1 b volume. In some embodiments, the ratio of trehalose to pullulan is in the range of 4:1 to 0.5:1 by weight, for example about 2:1. In some embodiments, the ratio of trehalose to pullulan is about 1:1. The PT solution is optionally made by dissolving the pullulan and trehalose into an additional volume of the same buffer used in the virus containing buffer.

In some embodiments, the virus is grown in a fetal bovine serum free media. In some embodiments, when the virus stock has protein, buffer exchange removes substantial amounts of the protein. As such, in some embodiments, the dialysed (i.e. buffer exchanged) solutions of the virus have minimal protein, typically less than 50 μg/mL and are considered albumin free, unless albumin is added to the composition of the application.

In some embodiments, the composition comprises albumin. In some embodiments, the albumin can be added, for example as human serum albumin (HAS), bovine serum albumin (BSA) or a recombinant albumin, for example recombinant human albumin. The pH of a composition may need to be adjusted to avoid a decrease in pH after adding albumin. In some embodiments, the concentration of the albumin in the composition is in the range of 0.125%-2.5%. In some embodiments, the concentration of the albumin in the composition is about 0.5% or about 2%. In some embodiments, albumin improves stability of the virus in the composition and/or reduces process loss in the method of preparing the dry composition. Lower concentrations of albumin, for example in the order of 0.05%, may be provided in some samples by way of carryover from the virus manufacturing process. While low concentrations of albumin from the carryover protein can improve stability and reduce process loss, these lower concentrations are not as effective as higher concentrations. In a commercial vaccine manufacturing process there should be no protein carryover and albumin can be added to the liquid compositions, for example in the range of 0.125%-2.5% described above, without adjusting for albumin carryover. BSA is used in examples described herein for convenience. However, in a human vaccine HSA or a recombinant human albumin may be used. In an animal vaccine, a form of albumin acceptable to the animal may be used. In some embodiments, the ratio of trehalose to albumin is from 3:1 to 25:1. In some embodiments, the ratio of trehalose to albumin is 10:1. In some embodiments, the albumin is present in the dry composition in a concentration of about 2 wt % to about 20 wt %. In some embodiments, the albumin is present in the dry composition in a concentration of about 2.5 wt %, about 4 wt %, about 7 wt %, or about 15 wt % and values therebetween.

In some embodiments, the composition comprises NaCl. NaCl may be present in the composition at a concentration of 25-150 mM or 50-100 mM. NaCl may be present in the composition at a concentration of 50 mM. Compositions with higher concentrations of NaCl (for example 250 mM) may have increased residual moisture, process loss or reduced storage stability. In some embodiments, NaCl was found to be beneficial in combination with a CM buffer but might not be beneficial in combination with a Tris-HCl buffer. In some embodiments, the NaCl is present in the dry composition in a concentration of about 1 wt % to about 12 wt %. In some embodiments, the NaCl is present in the dry composition in a concentration of about 1.5 wt %, about 2 wt %, about 3 wt %, about 4 wt %, about 6 wt %, or about 10 wt % and values thereretween.

An exemplary composition and an optional range of parts by mass (excluding water) for other compositions are described in some of the tables below. In some embodiments, the composition comprises about 23 to about 300 parts of the trehalose. In some embodiments, the composition comprises about 1 to about 10 parts of the buffer. In some embodiments, the composition comprises about 12 to about 150 parts pullulan. In some embodiments, the composition comprises about 2 to about 35 parts of the albumin. In some embodiments, the composition comprises about 2 to about 12 parts of the NaCl. In some embodiments, the composition comprises about 0.0001 to about 0.01 parts of the virus.

In some embodiments, in the method of the application the composition is dried with a step of foam drying. In some embodiments, foam drying includes a process that is carried out under vacuum sufficient to cause the composition to foam. In some embodiments, the foam drying is vacuum foam drying or vacuum foam freeze drying.

Drying temperature may have a material effect on at least one of residual moisture, process loss and storage stability. Drying at or near ambient temperature, for example about 20° C., produces low residual moisture, even in samples containing pullulan. As such, in some embodiments, at least part of the drying is carried out at a temperature of about 15° C. to about 40° C. In some embodiments, at least part of the drying is carried out at a temperature of about 1° C. to about 10° C. In some embodiments, the drying at a lower temperate at least initially, results in less process loss. In some embodiments, at least part of the drying is carried out at a temperature of about 20° C. In some embodiments, at least part of the drying is carried out at a temperature of about 4° C.

In some embodiments, the drying is carried out at two or more temperatures. In some embodiments, the drying is carried out at a temperature in the range of about −50° C. to about 15° C., or about 1° C. to about 10° C., optionally for 5-15 hours, followed directly or indirectly by drying at a higher temperature, for example about 15° C. to about 40° C., optionally for 5-15 hours. The method of the application may result in low residual moisture (RM), for example in the range of 1-10% or 1-7%, even in compositions with pullulan. As such, the method of the application achieves RM of about 1% to about 10%. As such, in some embodiments, the dry composition has a water content of less than 10 wt %. In some embodiments, the dry composition has a water content of less than 9 wt %. In some embodiments, the dry composition has a water content of less than 8 wt %. In some embodiments, the dry composition has a water content of less than 7 wt %. In some embodiments, the dry composition has a water content of less than 6 wt %. In some embodiments, the dry composition has a water content of less than 5 wt %. In some embodiments, the dry composition has a water content of about 1 wt % to about 10 wt %. In some embodiment, the dry composition a water content of about 1 wt % to about 9 wt %. In some embodiment, the dry composition has a water content of about 1 wt % to about 8 wt %. In some embodiment, the dry composition has a water content of about 1 wt % to about 7 wt %.

In some embodiments, the drying is carried out at a temperature in the range of about 1° C. to about 15° C., optionally for 5-15 hours, followed directly or indirectly by drying at a higher temperature of about 15° C. to about 40° C., optionally for 5-15 hours. In some embodiments, the drying further comprises precooling of the sample before the drying, optionally for 15 min to about 1 hour.

In some embodiments, all drying stages are carried out at a temperature above 0° C. Temperatures used herein are temperature settings of a drying cabinet, for example the temperature of a shelf in a freeze dryer. Temperature excursions of the composition itself below 0° C. caused by evaporative cooling of the composition, rather than by cooling the drying cabinet cabinet containing the composition, are not necessarily excluded. However, in some examples the composition is not frozen from a liquid prior to the application of a vacuum as in freeze drying. Storage stability of the dried product decreases with freeze drying in at least some compositions. Freeze drying may also result in dry product that is less stable when re-frozen during storage after drying. Optionally, temperature excursions of the composition itself below 0° C. caused by evaporative cooling of the composition may be avoided, for example by depressurizing a cabinet in stages.

In some embodiments, the drying is carried out in a lyophilizer or freeze dryer, either of which may be used without freezing, or a foam dryer. In some embodiments, the drying is carried out under vacuum sufficient to foam the composition, for example at a vacuum with a pressure less than 100 mBar or less than 10 mBar. In some examples, the vacuum pressure is less than 200 uBar. In some examples, the composition is placed in the vacuum as a bulk liquid rather than, for example, as a spray or thin film or as a frozen aqueous solid. In some embodiments, when the composition is dried using foam drying, this method produces a dry product in the form of a foam. In these embodiments, the composition may be transformed from a solution into a dried foam structure in one step involving simultaneous boiling or foaming, and evaporation. In this embodiment, vacuum sufficient to foam the composition is used. For aliquots of about 100 uL, the drying time may be in the range of 15-25 hours including a stage of secondary drying after a dried or glass foam is produced. Longer drying times, for example up to 48 hours, may also be used but does not appear to materially improve stability. Other drying times may be used for aliquots of different volumes.

In some embodiments, when the drying is carried out in a freeze dryer or other vacuum dryer operated at a temperature setpoint above 0 C. In some examples, despite the temperature setting of the dryer, as the cabinet is evacuated and the measured pressure drops to around 3-5 mBar, the temperature in the vials may drop below 0 C. In some embodiments, the excursion below 0 C is not a requirement of a process of the application. Unless stated otherwise, temperatures used in examples refer to the temperature setting of the dryer and not necessarily the temperature in the vials or the temperature of the composition.

In some embodiments the composition is stored in a nitrogen-enhanced atmosphere.

In some embodiments, the dried composition may be stored, for example at a temperature in the range of 1-55° C. or 1-40° C. or 4-25° C. In some embodiments, the virus is more stable in compositions stored at lower temperatures, for example 1-10° C. In some embodiments, compositions of the application, for example compositions comprising pullulan and trehalose, may preserve virus stability though temporary storage at a temperature below 0° C. In some embodiments, the composition of the application, for example the composition comprising trehalose and albumin and/or pullulan, preserves virus stability though temporary storage at a temperature above 0° C. In some embodiments, a composition of the application preserves virus stability at a temperature from about 2° C. to about 40° C., about 10° C. to about 30° C., or about 20° C. to about 25° C., or about 25° C. to about 40° C. In some embodiments, composition of the application, preserves virus stability for at least 2 weeks or at least 3 months at the above temperatures. In some embodiments, composition of the application, preserves virus stability for at least 4 days, or from 4 to 10 days at temperatures below freezing.

### III. Composition of the Application

The present application includes a composition comprising: a virus, trehalose, less than 10% water, a buffer, and optionally pullulan and/or albumin, for example at least 1% albumin by weight. In some embodiments, the virus is a virus in any recombinant, derived or modified form. In some embodiments, the virus is a live-attenuated virus, an inactivated virus, a viral vector or a recombinant virus. In some embodiments, the virus is an RNA virus, for example an enveloped RNA virus. In some embodiments, the virus is a vesicular stomatitis virus (VSV). In some embodiments, the VSV is a recombinant VSV. In some embodiments, the virus is derived from an adenovirus. In some embodiments, the virus is formulated for administration in a biological preparation. In some embodiments, the virus is formulated for administration as a vaccine.

The composition may provide a foamed glass (e.g. a sugar glass) encapsulating the virus. In some embodiments, the composition contains at least 90% by weight of trehalose, pullulan and albumin.

In some embodiments, the buffer is any buffer that maintains the pH of the composition of the application within the range of 6.8 to 8.2. In some embodiments, the buffer is a CM buffer or a Tris buffer, for example a Tris-HCl buffer. The buffer may include 10-50 mM of Tris. In some embodiments, the buffer maintains the pH of the composition in the range of 6.9 to 8.1 or 7.2-7.5. In some embodiments, the buffer is present in the dry composition at a concentration of about 0.5 wt % to about 10 wt %. In some embodiments, the buffer is present in the dry composition at a concentration of about 1 wt %, about 1.5 wt %, about 2 wt %, about 4 wt %, or about 8 wt % and values therebetween.

In some embodiments, other buffers, for example a Histidine buffer, may be used. In some embodiments, the buffer does not contain substantial amounts of crystal forming components. For example, phosphate-buffered saline (PBS) might reduce the performance of the composition of the application. In some embodiments, CM buffer may produce sulfate crystals and accordingly a Tris-HCl buffer may be preferred over a CM buffer in some examples. In some embodiments, the composition has less than 1 wt % on a fully dried basis of sulphate and phosphate salts.

In some embodiments, the trehalose is present in the composition at a concentration of about 1.25% (w/v) to about 15% (w/v). In some embodiments, the trehalose is present in the composition at a concentration of about 1.25% (w/v), about 2.5% (w/v), about 5% (w/v), or about 10% (w/v). Trehalose is available from a variety of commercial sources. In some embodiments, the trehalose is present in the dry composition in a concentration of about 30 wt % to about 70 wt %, or about 70 wt % to 99 wt %. In some embodiments, the trehalose is present in the dry composition in a concentration of about 35% wt %, about 35 wt %, about 40 wt %, about 45 wt %, about 50 wt %, about 55 wt %, about 60 wt %, about 65 wt %, or about 80 wt %, about 90 wt %, or about 95 wt % and values therebetween.

In some embodiments, the composition of the application comprises pullulan. In some embodiments the pullulan has a molecular weight in the range of about 100,000 to about 200,000. Pullulan having such molecular weights is commercially available. In some embodiments, the pullulan is present in the composition in a concentration of about 0.5% to about 15%. In some embodiments, the pullulan is present in the composition at a concentration of about 0.625%, about 1.25%, about 2.5%, about 5%, or about 10%. In some embodiments, the pullulan is present in the dry composition in a concentration of about 15% wt % to about 50 wt %. In some embodiments, the pullulan is present in the dry composition in a concentration of about 20 wt %, about 25 wt %, about 25 wt %, about 30 wt %, about 35 wt %, about 40 wt %, or about 45 wt % and values therebetween.

Optionally, different ratios of pullulan to trehalose may be used. PT solutions may be viscous and can be prepared independently of a virus containing buffer and then added to the virus containing buffer. A ratio of trehalose to pullulan may be in the range of 0.5:1 to 9:1. In some embodiments, the ratio of trehalose to pullulan is about 2:1. In some embodiments, the ratio of trehalose to pullulan is about 1:1. The PT solution is optionally made by dissolving the pullulan and trehalose into an additional volume of the same buffer that is used in the virus containing buffer.

In some embodiments, the virus is grown in a fetal bovine serum free media. In some embodiments, when the virus stock has protein, buffer exchange removes substantial amounts of the protein. As such, in some embodiments, the virus stock after buffer exchange has minimal protein, typically less than 50 μg/mL, and is considered albumin free. However, albumin may be added to the composition of the application.

In some embodiments, the composition of the application comprises albumin. In some embodiments, the albumin can be added, for example as human serum albumin (HSA), bovine serum albumin (BSA) or a recombinant albumin such as recombinant human albumin. The pH of a composition may need to be adjusted to avoid a decrease in pH after adding albumin. In some embodiments, the concentration of the albumin in the composition before drying is in the range of 0.125%-2.5%. In some embodiments, the concentration of the albumin in the composition before drying is about 0.5% or about 2%. BSA is used in examples described herein for convenience. However, in a human vaccine HSA or a recombinant human albumin may be used. In an animal vaccine, a form of albumin acceptable to the animal may be used. In some embodiments, the ratio of trehalose to albumin is from 3:1 to 25:1. In some embodiments, the ratio of trehalose to albumin is 10:1. In some embodiments, the albumin is present in the dry composition in a concentration of at least 1 wt %, for example about 2 wt % to about 20 wt %. In some embodiments, the albumin is present in the dry composition in a concentration of about 2.5 wt %, about 4 wt %, about 7 wt %, or about 15 wt % and values therebetween.

In some embodiments, the composition of the application comprises NaCl. NaCl may be present in the composition at a concentration of 25-150 mM or 50-100 mM. NaCl may be present in the composition at a concentration of 50 mM. Compositions with higher concentrations of NaCl (for example 250 mM) may have increased residual moisture, process loss or reduced storage stability. In some embodiments, NaCl was found to be beneficial in combination with a CM buffer but might not be beneficial in combination with a Tris-HCl buffer. In some embodiments, the NaCl is present in the dry composition in a concentration of about 1 wt % to about 12 wt %. In some embodiments, the NaCl is present in the dry composition in a concentration of about 1.5 wt %, about 2 wt %, about 3 wt %, about 4 wt %, about 6 wt %, or about 10 wt % and values thereretween.

In some embodiments, the composition is dried from a liquid composition or reconstituted into a liquid composition.

In some embodiments, the composition is a dry composition. In some embodiments, the dry composition is prepared by the method of the application. In some embodiments, the dry composition is a foam.

In some embodiments, the dry composition of the application has a water content of about 1% to about 10%. As such, in some embodiments, the dry composition has a water content of less than 10 wt %. In some embodiments, the dry composition has a water content of less than 9 wt %. In some embodiments, the dry composition has a water content of less than 8 wt %. In some embodiments, the dry composition has a water content of less than 7 wt %. In some embodiments, the dry composition has a water content of less than 6 wt %. In some embodiments, the dry composition has a water content of less than 5 wt %. In some embodiments, the dry composition has a water content of about 1 wt % to about 10 wt %. In some embodiment, the dry composition a water content of about 1 wt % to about 9 wt %. In some embodiment, the dry composition has a water content of about 1 wt % to about 8 wt %. In some embodiment, the dry composition has a water content of about 1 wt % to about 7 wt %.

An exemplary composition and an optional range of parts by mass (excluding water) for other compositions are described in Table 1 or other tables below. In some embodiments, the dry composition comprises about 23 to about 300 parts of the trehalose. In some embodiments, the dry composition comprises about 1 to about 10 parts of the buffer. In some embodiments, the dry composition comprises about 12 to about 150 parts pullulan. In some embodiments, the dry composition comprises about 2 to about 35 parts of the albumin. In some embodiments, the dry composition comprises about 2 to about 12 parts of the NaCl. In some embodiments, the dry composition comprises about 0.0001 to about 0.01 parts of the virus.

In some embodiments, the dried composition may be stored, for example at a temperature in the range of 1-55° C. or 1-40° C. or 4-25° C. In some embodiments, the virus is more stable in compositions stored at lower temperatures, for example 1-10° C. In some embodiments, compositions of the application, for example compositions comprising pullulan and trehalose, may preserve virus stability though temporary storage at a temperature below 0° C. In some embodiments, the composition of the application, for example the composition comprising pullulan, trehalose and albumin, preserves virus stability though temporary storage at a temperature above 0° C. In some embodiments, the composition of the application preserves virus stability at a temperature from about 2° C. to about 40° C., about 10° C. to about 30° C., or about 20° C. to about 25° C., or about 25° C. to about 40° C. In some embodiments, composition of the application, preserves virus stability for at least 2 weeks at the above temperatures. In some embodiments, composition of the application, preserves virus stability for at least 4 days, or from 4 to 10 days at temperatures below freezing.

In some embodiments, the composition of the application is used as, or as part of, or as a precursor of, a vaccine.

In some embodiments, the vaccine is for intramuscular, subcutaneous, intradermal, transdermal, oral, peroral, nasal, and/or inhalative application.

In some embodiments, the vaccine can be prepared by blending the composition of the application with one or more pharmaceutically acceptable excipients to generate a vaccine formulation. Exemplary pharmaceutically acceptable excipients for the purposes of pharmaceutical compositions disclosed herein include, but are not limited to, binders, disintegrants, superdisintegrants, lubricants, diluents, fillers, flavors, glidants, sorbents, solubilizers, chelating agents, emulsifiers, thickening agents, dispersants, suspending agents, adsorbents, granulating agents, buffers, coloring agents and sweeteners or combinations thereof.

TABLE 1

| | | | | | Total | | Range |
|---|---|---|---|---|---|---|---|
| | Concen- | | | ug/100 | mass | Percent | in Parts |
| Component | tration | Vol | MW | uL | (ug) | Film | by Mass |
| Tris-HCl | 10 mM | 100 | 121.14 | | 121.14 | 1.533 | 1-10 |
| MgS04 | 10 mM | 100 | 120.336 | | 120.336 | 1.523 | 0-5 |
| CaCl2 | 10 mM | 100 | 110.98 | | 110.98 | 1.405 | 0-5 |
| Gelatin | 0.005% | 100 | | 5 | 5 | 0.063 | 0-2 |
| Albumin | 0.50% | 100 | | 500 | 500 | 6.329 | 2-35 |
| NaCl | 50 mM | 100 | 58.44 | | 292.2 | 3.699 | 2-12 |
| Pullulan | 2.50% | 90 | | 2500 | 2250 | 28.482 | 0-150 |
| Trehalose | 5% | 90 | | 5000 | 4500 | 56.964 | 25-300 |
| Virus | 1.00E+08 | | 265 mDa | | 0.04 | 0.001 | .0001-.01 |
| Total | | | | | 7899.7 | 100 | |

US Patent Application Publication No. US 2019/0111006 A1, Method of Long-Term Preservation of Chemical and Biological Species Using Sugar Glasses, is incorporated herein in its entirety by this reference.

In the examples presented below, modified reporter viruses are used as a model to represent recombinant viruses that would be used in viral vector vaccines, in particular VSV or AdV vectored vaccines. The VSV are Indiana serotype (e.g. VSV-XN GFP), replicating and have modification to produce green fluorescent protein (GFP) (e.g. VSV-XN GFP) or a red fluorescent protein (mCherry). The AdV are non-replicating human adenovirus serotype 5 with modifications to delete the E1/E3 genes and to produce GFP (huAd5-GFP). Considering the dilutions in the assays, results are typically considered to be +/−0.25 log unless indicated otherwise.

### EXAMPLES

### Example 1: Formulation Preparation

### Example 1a: Tris/Gelatin/Sorbitol Formulation with Vesicular Stomatitis Virus-Green Fluorescent Protein (VSV-GFP)

VSV-GFP was transferred from an initial commercial sample to a buffer solution by way of buffer exchange using a spin column to create a stock solution. The buffer was a tris(hydroxymethyl)aminomethane (Tris) buffer (10 mM) with a pH of 7.2. A storage solution of 0.5% gelatin and 2% sorbitol mixed into the same Tris buffer was prepared. 90 μL of the storage solution was mixed with 10 uL of the stock solution to create 100 μL aliquots having an initial titer of about 10⁹ PFU.

### Example 1b: Pullulan/Trehalose/CM Formulation with VSV-mCherry

VSV-mCherry was transferred from an initial commercial sample to a buffer solution by way of buffer exchange using a spin column to create a stock solution. The buffer was a CM buffer with a pH of 7.2. A storage solution of 5% pullulan and 10% trehalose mixed into the same CM buffer was prepared. 90 μL of the storage solution was mixed with 10 μL of the stock solution to create 100 μL aliquots having an initial titer of about 10⁹ PFU. VSV-mCherry had minimal albumin carryover of about 10 μg, or about 0.01% of the storage solution.

### Example 1c: Pullulan/Trehalose/CM Formulation with VSV-GFP

VSV-GFP was transferred from an initial commercial sample to a buffer solution by way of buffer exchange using a spin column to create a stock solution. The buffer was a CM buffer with a pH of 7.2. A storage solution of 5% pullulan and 10% trehalose mixed into the same CM buffer was prepared. 90 μL of the storage solution where mixed with 10 μL of the stock solution to create 100 μL aliquots having an initial titer of about 10⁹ PFU. VSV-GFP had albumin carryover of about 60 μg, or about 0.05% of the storage solution.

### Example 1d: Pullulan/Trehalose/CM/Albumin Formulation with VSV-mCherry and Albumin

VSV-mCherry was transferred from an initial commercial sample to a buffer solution by way of buffer exchange using a spin column to create a stock solution. The buffer was a CM buffer with a pH of 7.2. A storage solution of 2.5% pullulan, 5% trehalose and 0.5% albumin mixed into the same CM buffer was prepared. 90 μL of the storage solution was mixed with 10 μL of the stock solution to create 100 μL aliquots having an initial titer of about 10⁹ PFU.

### Example 1e: Pullulan/Trehalose/CM/BSA/NaCl Formulation with VSV-GFP

VSV-GFP was transferred from an initial commercial sample to a buffer solution by way of buffer exchange using a spin column to create a stock solution. The buffer was a CM buffer modified by adding 0.5% bovine serum albumin (BSA) and 50 mM NaCl with a pH of 7.2. A storage solution of 2.5% pullulan and 5% trehalose mixed into the same modified CM buffer was prepared. 90 μL of the storage solution was mixed with 10 μL of the stock solution to create 100 μL aliquots having an initial titer of about 10⁹ PFU.

### Example 1f: Tris/Trehalose Formulation with VSV-GFP

VSV-GFP was transferred from an initial commercial sample to a buffer solution by way of buffer exchange using a spin column to create a stock solution. The buffer was a Tris buffer (10 mM) with a pH of 7.2. A storage solution of 5% trehalose mixed into the same Tris buffer was prepared. 90 μL of the storage solution was mixed with 10 μL of the stock solution to create 100 μL aliquots having an initial titer of about 10⁹ PFU.

### Example 2: Tris/Gelatin/Sorbitol Composition with VSV-GFP Dried at 20° C.

Aliquots of the composition of Example 1a were dried at 20° C. for 21 hours. Aliquots dried at 200 μBar (A) had an average residual moisture (RM) of 2.3%. Aliquots dried at 16 μBar (B) had an average residual moisture (RM) of 1.1%.

Aliquots A and B both experienced a process loss in titer of about 1 log, as determined by a plaque assay performed directly after drying. There were no detectable PFU after storing the dried composition for 7 days at 37° C.

As indicated by these results, the combination of Tris, gelatin and sorbitol was not effective at stabilizing the composition containing VSV.

### Example 3: Compositions of Examples 1b, 1c and 1d Dried at 20° C.

Aliquots of the composition of Examples 1b, 1c and 1d were dried at 20° C. under vacuum for 24 hours and stored at 37° C. Process loss and titer loss (including process loss) at 7 and 18 days is described in Table 1 below.

TABLE 2
Process and Titer Loss

| Composition | Example 1b | Example 1c | Example 1d |
|---|---|---|---|
| Process loss (log) | 2.0 | 1.5 | 1.0 |
| Titer Loss Day 7 (log) | 2.5 | 2.1 | 1.7 |
| Titer Loss Day 18 (log) | 4 | 2.5 | 2.1 |

### Example 4: PT/CM/BSA/NaCl Composition with VSV-GFP Dried at 20° C.

Aliquots of the composition in Example 1e were dried at 20° C. for 21 hours. Aliquots dried at 200 μBar (A) had an average residual moisture (RM) of 3.9%. Aliquots dried at 16 μBar (B) had an average residual moisture (RM) of 3.5%.

Aliquots A experienced an average process loss in titer of 1.3 log. Aliquots B experienced an average process loss in titer of about 1 log.

Aliquots B were stored at 37° C. Plaque assays performed at 7 and 14 days indicated a loss in titer (relative to the initial 10⁹ PFU sample titer) of about 2 log at both times. Plaque assays performed at 28 days indicated a loss in titer (relative to the initial 10⁹ PFU sample titer) of about 3 log.

### Example 5: Tris/Gelatin/Sorbitol Composition with VSV-GFP Dried at 4° C.

Aliquots of the composition of Example 1a were dried for 21 hours at 4° C. and about 12-15 μBar. The average residual moisture (RM) of the dried compositions was 0.7%. Drying produced a process loss in titer of about 0.3 log, as determined by a plaque assay.

Aliquots of the composition were stored at 37° C. Plaque assays performed at 7 and 14 days indicated losses in titer (relative to the initial 10⁹ PFU sample titer) of about 5.4 log and 8 log respectively.

### Example 6: Pullulan/Trehalose (PT)/CM/BSA/NaCl Composition with VSV-GFP Dried at 4° C.

Aliquots of the composition of Example 1e were dried for 21 hours at 4° C. and about 12-15 μBar. The average residual moisture (RM) of the dried compositions was 8.6%. Drying produced a process loss in titer of about 0.4 log, as determined by a plaque assay.

Aliquots of the composition were stored at 37° C. Plaque assays performed at 7 and 14 days indicated losses in titer (relative to the initial 10⁹ PFU sample titer) of about 2.5 log and 3 log respectively.

### Example 7: PT/CM/BSA/NaCl Composition with VSV-GFP Dried at 4° C./25° C.

Aliquots of the composition of Example 1e were dried for 21 hours at about 15 μBar. The 21 hours of drying included 10 hours at 4° C., followed by a temperature ramping up from 4° C. to 25° C. over 5 hours, followed by drying for 6 hours at 25° C. The average residual moisture (RM) of the dried compositions was 6.1%. Drying produced no detectable process loss, as determined by a plaque assay.

Aliquots of the composition were stored at 37° C. Plaque assays performed at 7 and 14 days indicated losses in titer (relative to the initial 10⁹ PFU sample titer) of about 1.2 log and 1.8 log respectively.

### Example 8: Tris/Trehalose Composition with VSV-GFP Dried at 4° C./25° C.

Aliquots of the composition of Example 1f were dried for 21 hours at about 15 μBar. The 21 hours of drying included 10 hours at 4° C., followed by a temperature ramping up from 4° C. to 25° C. over 5 hours, followed by drying for 6 hours at 25° C. The average residual moisture (RM) of the dried compositions was 3.5%. Drying produced a process loss in titer of about 0.5 log, as determined by a plaque assay.

Aliquots of the composition were stored at 37° C. Plaque assays performed at 7 and 14 days indicated losses in titer (relative to the initial 10⁹ PFU sample titer) of about 1.1 log and 1.2 log respectively.

### Example 9: PT/CM/BSA/NaCl Composition with VSV-GFP Dried at −50° C./25° C.

Aliquots of the composition of Example 1e were dried for 21 hours at about 15 μBar. The 21 hours of drying included 10 hours at −50° C., followed by a temperature ramping up from −50° C. to 25° C. over 6 hours, followed by drying for 5 hours at 25° C. The average residual moisture (RM) of the dried compositions was 1.7%. Drying produced about 0.5 log process loss, as determined by a plaque assay.

Aliquots of the composition were stored at 37° C. A plaque assays performed at 7 days indicated a loss in titer (relative to the initial 10⁹ PFU sample titer) of about 3 log.

### Example 10: Tris/Trehalose Composition with VSV-GFP Dried at −50° C./25° C.

Aliquots of the composition of Example 1f were dried for 21 hours at about 15 μBar. The 21 hours of drying included 10 hours at −50° C., followed by a temperature ramping up from −50° C. to 25° C. over 6 hours, followed by drying for 5 hours at 25° C. The average residual moisture (RM) of the dried compositions was 1.0%. Drying produced a process loss in titer of about 1 log, as determined by a plaque assay.

Aliquots of the composition were stored at 37° C. A plaque assay performed at 7 days indicated a loss in titer (relative to the initial 10⁹ PFU sample titer) of about 8 log.

### Example 11: PT/CM/BSA/NaCl Composition with VSV-GFP Dried at 4° C./37° C.

Aliquots of the composition of Example 1e were dried for 21 hours at about 15 μBar. The 21 hours of drying included 10 hours at 4° C., followed by a temperature ramping up from 4° C. to 37° C. over 6 hours, followed by drying for 6 hours at 37° C. The average residual moisture (RM) of the dried compositions was about 5.7%. Drying produced a process loss of about 0.5 log.

Aliquots of the composition were stored at 37° C. Plaque assay performed at 7 days indicated loss in titer (relative to the initial 10⁹ PFU sample titer) of about 1.5 log. Plaque assay performed at 14 days indicated loss in titer (relative to the initial 10⁹ PFU sample titer) of about 1.9 log.

### Example 12-PT/CM/BSA/NaCl Composition Dried at 20° C.

VSV-GFP was transferred from an initial solution to a dialysis buffer solution by way of buffer exchange using Zeba spin column to create two stock solutions.

Two formulations were prepared using 10% by volume of stock solution and 90% by volume of a solution of either a) pullulan and trehalose or b) gelatin and sorbitol, each dissolved in one of the dialysis buffers, as indicated in Table 3 below.

TABLE 3
Formulations for the Study

| | |
|---|---|
| E-a | Control |

| | |
|---|---|
| VSV Stock | GFP (Lot 354) |

| | | |
|---|---|---|
| Dialysis Buffer | CM + 0.5% BSA + 50 | Tris |
| (pH 7.2) | mM NaCl | 0.5% Gelatin + |
| Formulation (Dialysis | 2.5% pullulan and | 2% Sorbitol |
| Buffer Base) | 0.125M (5%) trehalose | |

| | |
|---|---|
| Total Volume | 100 |

100 uL aliquots of the samples were foam dried under vacuum in 2 mL glass vials for 21 hours at 20° C. Some of the samples were dried in a dessicator at a pressure setpoint of 200 μBar. Other samples were dried with freeze dryer at a pressure setpoint of 16 μBar. No statistically significant difference in stability was detected between samples dried at the different pressures. After drying, samples were stoppered and crimped.

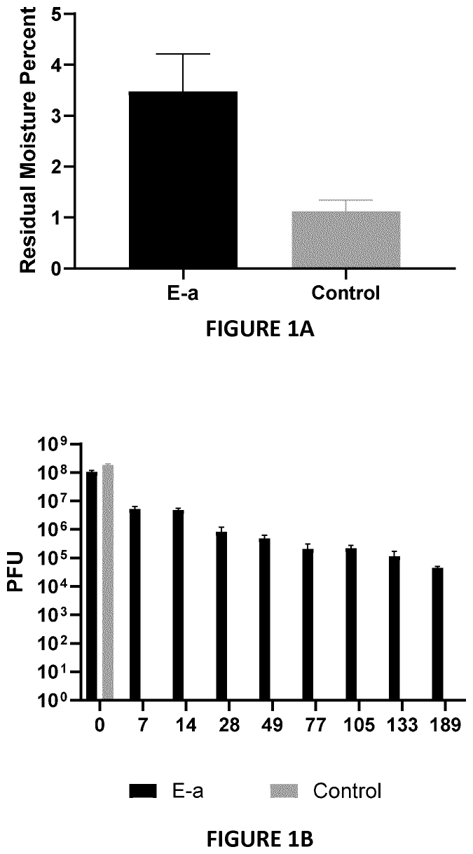

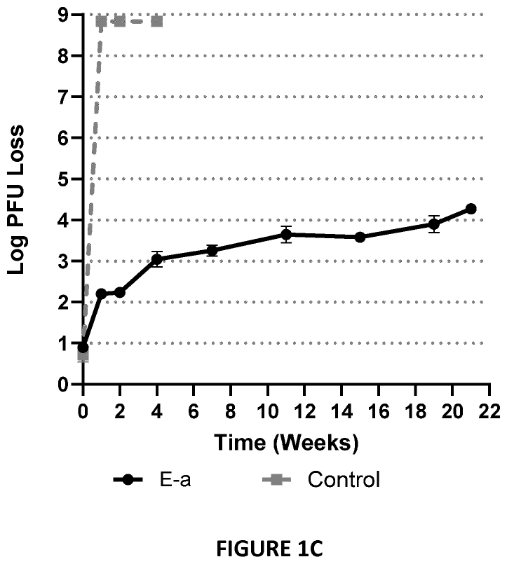

The residual moisture (RM) of the E-a formulation was about 3.5%. The residual moisture of the control (comparative) formulation was about 1.1% (FIG. 1A). There was less than 1 log of process loss for both formulation samples

The samples were incubated at 37° C. for 22 and 27 weeks (FIGS. 1B and 1C). The control formulation had no detectable PFU after 7 days of incubation at 37° C. E-a formulation showed about 2.5 log total loss (2×10⁵ PFU) at week 15 and about 4.3 log total loss at week 27. Table 4 below shows the wt % of the components in the dry formulation.

TABLE 4

| | E-a (As made Formulation) | | E-a Wt % Dried Formulation |
|---|---|---|---|
| Pullulan | 2.5 | wt % | 28.5 |
| Trehalose | 5 | wt % | 57.0 |
| BSA | 0.5 | wt % | 6.3 |
| NaCl | 50 | mM | 3.7 |
| Tris | 10 | mM | 1.5 |
| Gelatin | 0.01 | wt % | 0.1 |
| MgSO4 | 10 | mM | 1.5 |
| CaCl2 | 10 | mM | 1.4 |

| | | |
|---|---|---|
| Virus | 1e8 particles | 0.001 |

### Example 13: PT/CM/BSA/NaCl Composition Dried at 4° C./25° C.

VSV-GFP was transferred from an initial solution to a dialysis buffer solution by way of buffer exchange using Zeba spin column (0.5 mL columns) to create two stock solutions.

Two formulations were prepared using 10% by volume of stock solution and 90% by volume of a solution of either a) pullulan and trehalose or b) trehalose, each dissolved in one of the dialysis buffers, as indicated in Table 4 below.

TABLE 4
Formulations for The Study

| | E-b | Trehalose |
|---|---|---|
| VSV Stock | GFP (Lot 356) | |
| Dialysis Buffer (pH 7.2) | CM + 0.5% BSA + | Tris |
| | 50 mM NaCl | |
| Formulation (+Dialysis Buffer) | 2.5% Pullulan | 5% Trehalose |
| | 0.125M (5%) | |
| | Trehalose | |

| | |
|---|---|
| Total Volume | 100 |

100 uL aliquots of the samples were foam dried in freeze dryer in 2 mL glass vials for 21 hours using a two-stage drying protocol. The first (primary) drying stage was 10 hours at 4° C. The second stage was 4.5 hours at 25° C. Between the first and second stage, the temperature ramped from 4° C.-25° C. over 6.5 hours. The pressure setpoint during the entire 21 hours was about 16 μBar. After drying, samples were stoppered and crimped.

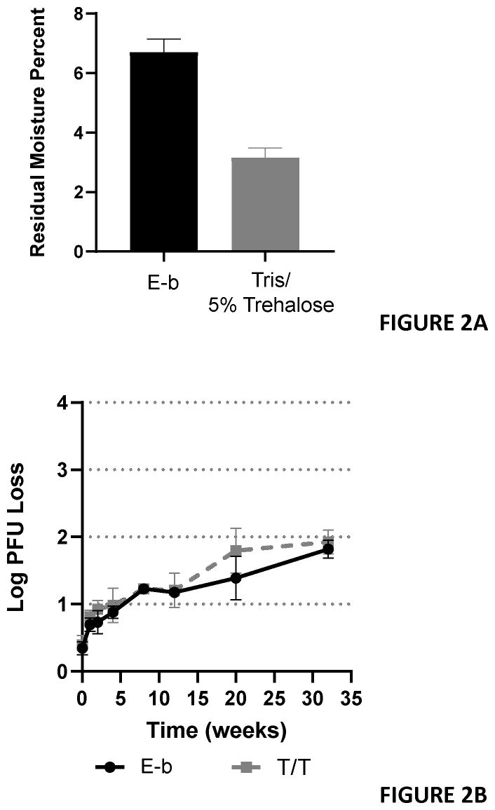

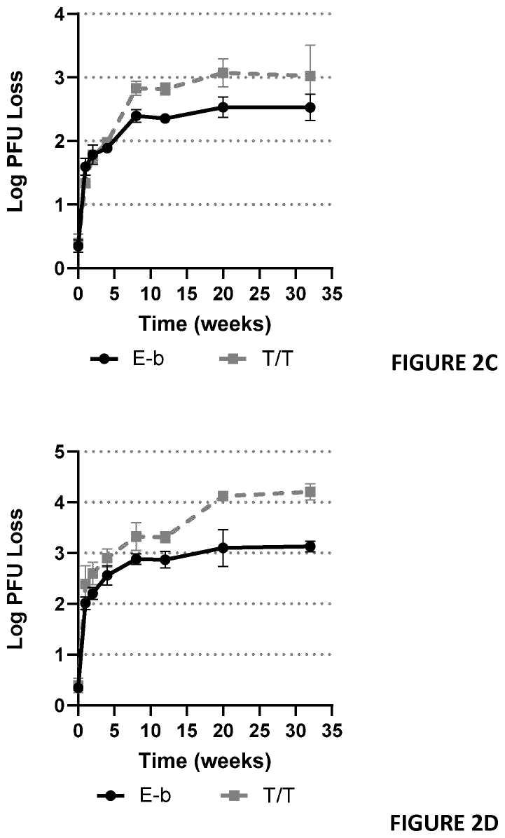

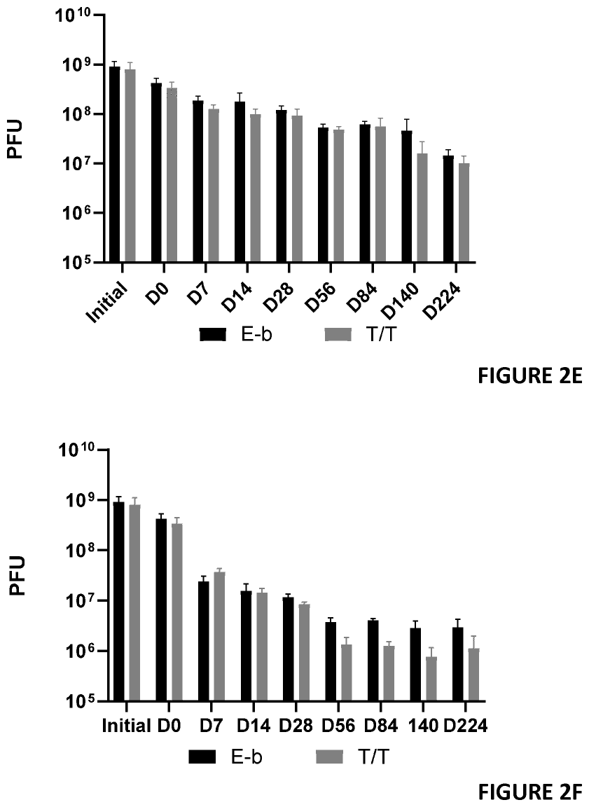

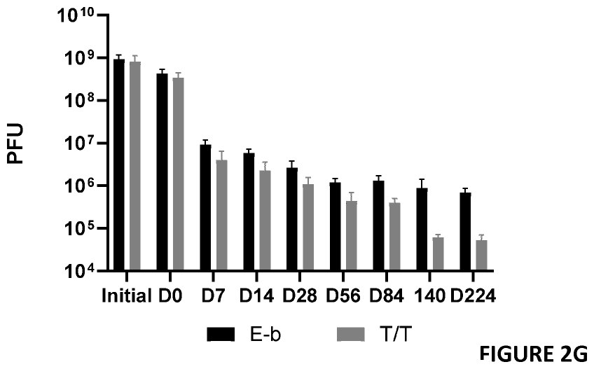

The residual moisture (RM) of the E-b formulation was about 6.7%. The residual moisture of the Trehalose formulation was about 3.2% (FIG. 2A). There was less than 1 log of process loss for both samples.

The samples were incubated at 4° C., 25° C. and 37° C. for 32 weeks. FIGS. 2B-G present the results of this study. The results show similar results in E-b and Trehalose formulations in the 4° C. incubated samples. E-b formulation showed less total loss at week 32 at higher temperatures. Table 5 below shows the wt % of the components in the dry formulation.

TABLE 5

| | | | | Tris/ | | Tris/ |
|---|---|---|---|---|---|---|
| | E-b | | E-b | Trehalose | | Trehalose |
| | (As made | | Wt % Dried | (As Made | | (Wt % Dried |
| | Formulation) | | Formulation | Formulation) | | Formulation) |
| Pullulan | 2.5 | wt % | 28.5 | — | | — |
| Trehalose | 5 | wt % | 57.0 | 5 | wt % | 97.4 |
| BSA | 0.5 | wt % | 6.3 | — | | — |
| NaCl | 50 | mM | 3.7 | — | | — |
| Tris | 10 | mM | 1.5 | 10 | mM | 2.5 |

| | | | | | |
|---|---|---|---|---|---|
| Gelatin | 0.01 | wt % | 0.1 | — | — |
| MgSO4 | 10 | mM | 1.5 | — | — |
| CaCl2 | 10 | mM | 1.4 | — | — |

| | | | | |
|---|---|---|---|---|
| Virus | 1e8 particles | 0.001 | 1e8 particles | 0.001 |

### Example 14: PT/CM/BSA/NaCl Composition in FBS Free Media Dried at 4° C./25° C. (Sprint 19)

In the examples described above, VSV-GFP stock was grown in fetal bovine serum (FBS). After buffer exchange some albumin carried over into the VSV stock. In this example and the examples described below, VSV-GFP is grown under GMP conditions in FBS free media. While there is still some protein in the VSV stock, after buffer exchange the stock solutions have minimal protein, typically less than 50 μg/mL. Formulations made from these stock solutions are considered albumin free unless albumin is added.

FBS free VSV-GFP was transferred from an initial solution to a dialysis buffer solution by way of buffer exchange using a Zeba spin column (0.5 mL columns) to create two stock solutions.

Four formulations were prepared using 10% by volume of stock solution and 90% by volume of a formulation solution, each dissolved in one of the dialysis buffers, as indicated in Table 6 below.

TABLE 6
Formulations for The Study

| | E-c1 | E-c1/−BSA | Trehalose + BSA | Trehalose − BSA |
|---|---|---|---|---|
| VSV Stock | GFP (Lot 357) | | | |
| Dialysis Buffer | CM + 50 mM | CM + 50 mM NaCl | Tris + 0.5% | Tris |
| (pH 7.2) 10 u: | NaCl + 0.5% BSA | | BSA | |
| Formulation | 2.5% Pullulan | 2.5% Pullulan | 5% Trehalose | 5% Trehalose |
| (+Dialysis Buffer) | 5% (0.125M) | 5% (0.125M) | | |
| 90 uL | Trehalose | Trehalose | | |

| | |
|---|---|
| Total Volume | 100 uL |

100 uL aliquots of the samples were foam dried in freeze dryer in 2 mL glass vials for 21 hours using a two-stage drying protocol. The first (primary) drying stage was 10 hours at 4° C. The second stage was 4.5 hours at 25° C. Between the first and second stage, the temperature ramped from 4-25° C. over 6.5 hours. The samples were pre-cooled to 4° C. for 30 minutes before starting the drying. The pressure setpoint during the entire 21 hours was about 16 μBar. After drying, samples were stoppered and crimped.

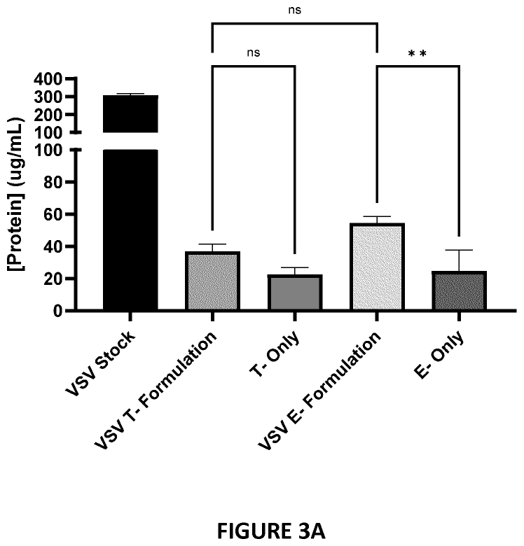

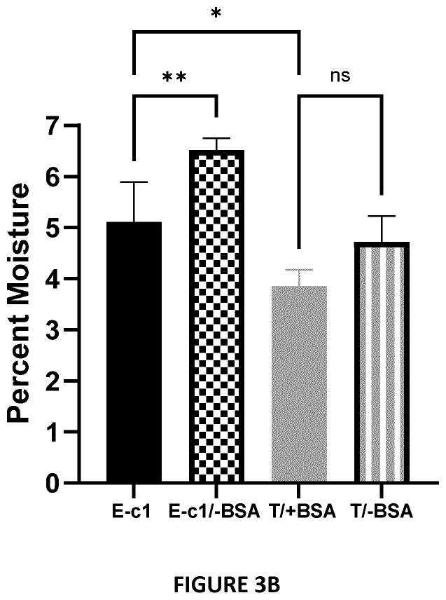

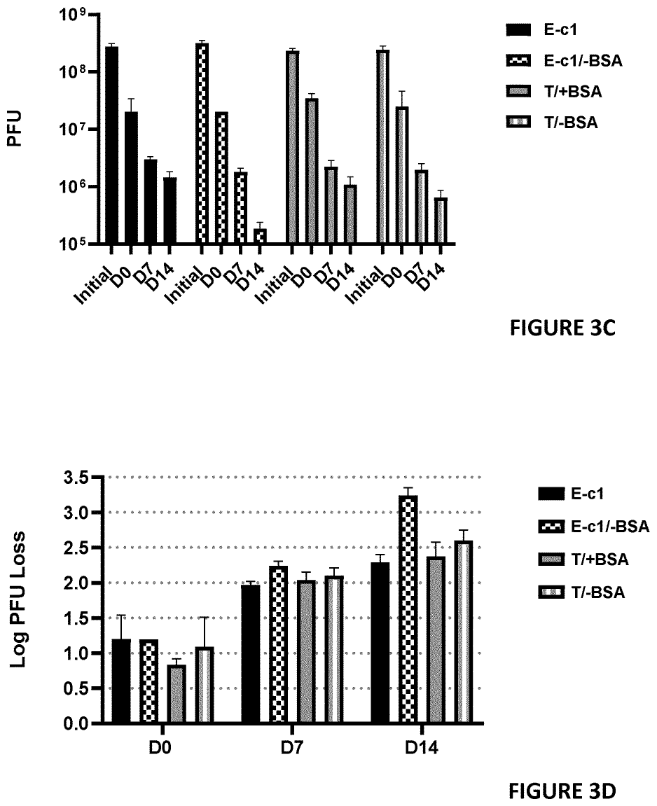

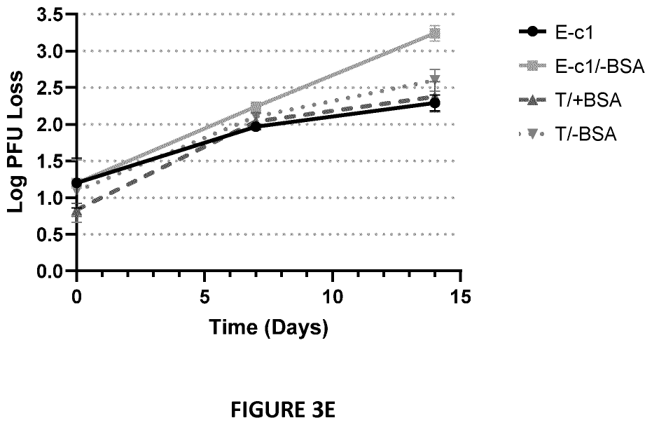

The samples were analyzed to quantify total protein using Bradford analysis and the results are shown in FIG. 3A. Background protein was detected in both buffers alone (T-Only and E-Only). Protein levels in dialyzed (buffer-exchanged) formulations (VSV T-Formulation and VSV E-Formulation) had insignificant protein levels.

The residual moisture (RM) of the E-c formulations was about 5% with BSA added and 6% without BSA. The residual moisture of the Trehalose formulation was about 4% with BSA and 5% without BSA. FIG. 3B presents the results.

The samples were incubated at 37° C. for 14 days. FIGS. 3C-3E presents the results of this study. The results showed no difference in titer between formulations post dialysis. Pre-dialysis calculated titer was 2.3e10 PFU/mL. Post dialysis for all samples was ˜2.5e10 PFU/mL. The results show improvement in stability in formulations with albumin. Table 7 below shows the wt % of the components in the dry formulation.

TABLE 7

| | | | Elarex | Elarex | | Elarex | Tris/ | | Tris/ | | | |
|---|---|---|---|---|---|---|---|---|---|---|---|---|
| | Elarex | | Lead | Lead − | | Lead − | Trehalose + | | Trehalose + | Tris/ | | Tris/ |
| | Lead | | Wt % | BSA | | BSA | BSA | | BSA | Trehalose | | Trehalose |
| | (As made | | Dried | (As made | | Wt % Dried | (As made | | Wt % Dried | (As made | | (Wt % Dried |
| | Formulation) | | Formulation | Formulation) | | Formulation | Formulation) | | Formulation | Formulation) | | Formulation) |
| Pullulan | 2.5 | wt % | 28.5 | 2.5 | wt % | 30.4 | — | | — | — | | — |
| Trehalose | 5 | wt % | 57.0 | 5 | wt % | 60.8 | 5 | wt % | 87.9 | 5 | wt % | 97.4 |
| BSA | 0.5 | wt % | 6.3 | — | | — | 0.5 | wt % | 9.8 | — | | — |
| NaCl | 50 | nM | 3.7 | 50 | mM | 3.9 | — | | — | — | | — |
| Tris | 10 | nM | 1.5 | 10 | mM | 1.6 | 10 | mM | 2.4 | 10 | mM | 2.5 |

| | | | | | | | | | | |
|---|---|---|---|---|---|---|---|---|---|---|
| Gelatin | 0.01 | wt % | 0.1 | 0.01 | wt % | 0.1 | — | — | — | — |
| MgSO4 | 10 | mM | 1.5 | 10 | mM | 1.6 | — | — | — | — |
| CACl2 | 10 | mM | 1.4 | 10 | mM | 1.5 | — | — | — | — |

| | | | | | | | | |
|---|---|---|---|---|---|---|---|---|
| Virus | 1e8 | 0.001 | 1e8 | 0.001 | 1e8 | 0.001 | 1e8 | 0.001 |
| | particles | | particles | | particles | | particles | |

### Example 15: PT/CM/BSA/NaCl Composition in FBS Free Media Dried at 4° C./25° C. With 0.5%/2% BSA

FBS free VSV-GFP was transferred from an initial solution to a dialysis buffer solution by way of buffer exchange using a Zeba spin column (0.5 mL columns) to create stock solutions.

Eight formulations were prepared using 10% by volume of stock solution and 90% by volume of a formulation solution, each dissolved in one of the dialysis buffers, as indicated in Table 6 below.

TABLE 6
Formulations for The Study

| | E-c1 | | E-c2 | | E-c3 | | E-c4 | | E-c5 | | E-c6 | | E-c7 | | E-c8 | |
|---|---|---|---|---|---|---|---|---|---|---|---|---|---|---|---|---|
| VSV Stock | GFP (Lot 357) | | | | | | | | | | | | | | | |
| Dialysis Buffer | CM + 0.5% BSA + 50 mM NaCl | | | | | | | | CM + 2% BSA + 50 mM NaCl | | | | | | | |
| Formulation | 2.5% | P | 5% | P | 5% | P | 10% | P | 2.5% | P | 5% | P | 5% | P | 10% | P |
| | 5% | T | 10% | T | 5% | T | 10% | T | 5% | T | 10% | T | 5% | T | 10% | T |

| | |
|---|---|
| Total Volume | 100 |

“P” indicates pullulan and “T” indicates trehalose

100 uL aliquots of the samples were foam dried in freeze dryer 2 mL glass vials for 21 hours using a two-stage drying protocol. The first (primary) drying stage was 10 hours at 4° C. The second stage was 4.5 hours at 25° C. Between the first and second stage, the temperature ramped from 4-25° C. over 6.5 hours. The samples were pre-cooled to 4° C. for 30 minutes before starting the drying. The pressure setpoint during the entire 21 hours was about 16 μBar. After drying, samples were stoppered and crimped.

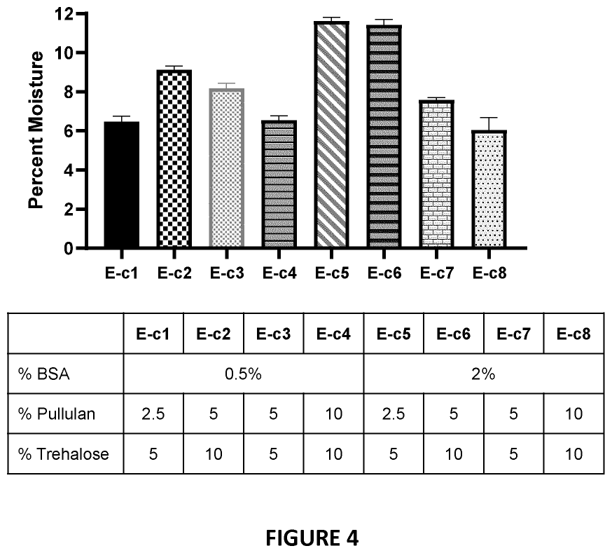

FIG. 4 presents the RM of the samples.

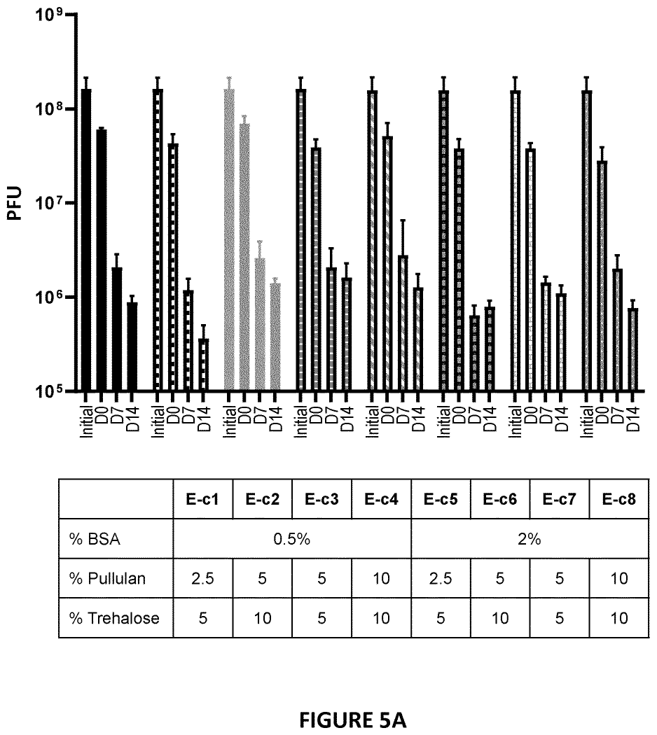

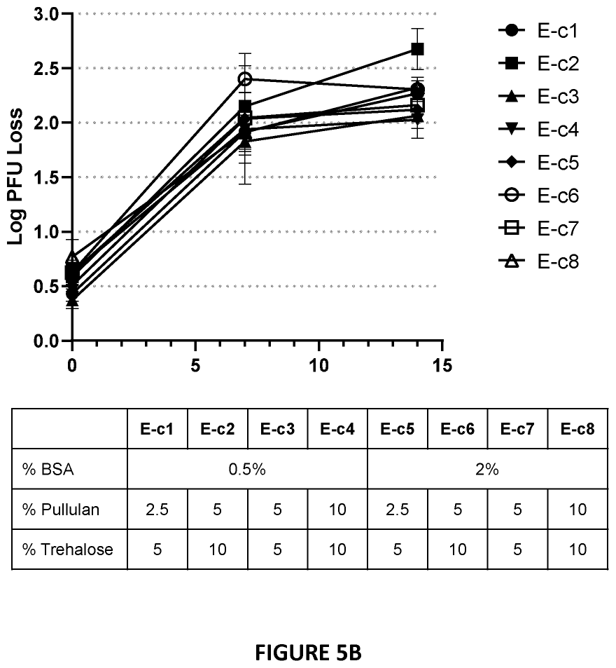

The samples were incubated at 37° C. for 7 days. FIGS. 5A-5B present the results of this study. Table 8 below shows the wt % of the components in the dry formulation.

TABLE 8

| | E-c1 | | E-c1 | E-c2 | | E-c2 | E-c3 | | E-c3 | E-c4 | | E-c4 |
|---|---|---|---|---|---|---|---|---|---|---|---|---|
| | (As made | | Wt % Dried | (As made | | Wt % Dried | (As made | | Wt % Dried | (As made | | (Wt % Dried |
| | Formulation) | | Formulation | Formulation) | | Formulation | Formulation) | | Formulation | Formulation) | | Formulation) |
| Pullulan | 2.5 | wt % | 28.5 | 5 | wt % | 30.7 | 5 | wt % | 44.3 | 10 | wt % | 47.0 |
| Trehalose | 5 | wt % | 57.0 | 10 | wt % | 61.4 | 5 | wt % | 44.3 | 10 | wt % | 47.0 |
| BSA | 0.5 | wt % | 6.3 | 0.5 | wt % | 43.4 | 0.5 | wt % | 4.9 | 0.5 | wt % | 2.6 |
| NaCl | 50 | mM | 3.7 | 50 | mM | 2.0 | 50 | mM | 2.9 | 50 | mM | 1.5 |
| Tris | 10 | mM | 1.5 | 10 | mM | 0.8 | 10 | mM | 1.2 | 10 | mM | 0.6 |
| Gelatin | 0.01 | wt % | 0.1 | 0.01 | wt % | 0.03 | 0.01 | wt % | 0.05 | 0.01 | wt % | 0.03 |
| MgSO4 | 10 | mM | 1.5 | 10 | mM | 0.8 | 10 | mM | 1.2 | 10 | mM | 0.6 |
| CaCl2 | 10 | mM | 1.4 | 10 | mM | 0.8 | 10 | mM | 1.1 | 10 | mM | 0.6 |

| | | | | | | | | |
|---|---|---|---|---|---|---|---|---|
| Virus | 1e8 | 0.001 | 1e8 | 0.0003 | 1e8 | 0.0004 | 1e8 | 0.0002 |
| | particles | | particles | | particles | | particles | |

### Example 16: PT/CM/BSA/NaCl Composition Dried at 4° C./25° C. With Nitrogen Backfill

Serum free VSV-GFP was transferred from an initial solution to a dialysis buffer solution by way of buffer exchange using a spin column to create two stock solutions.

Two formulations were prepared using 10% by volume of stock solution and 90% by volume of a solution of either a) pullulan and trehalose or b) trehalose, each dissolved in one of the dialysis buffers, as indicated in Table 9 below.

TABLE 9
Formulations for The Study

| | |
|---|---|
| E-c1 | Trehalose |

| | |
|---|---|
| VSV Stock | GFP (Lot 357) |

| | | |
|---|---|---|
| Dialysis Buffer | CM + 0.5% BSA + 50 | Tris |
| (pH 7.2) | mM NaCl | |
| Formulation | 2.5% Pullulan | 5% Trehalose |
| (+Dialysis Buffer) | 5% (0.125M) Trehalose | |

| | |
|---|---|
| Total Volume | 100 |

100 uL aliquots of the samples were foam dried in freeze dryer 2 mL glass vials for 19.5 hours using a two-stage drying protocol. The first (primary) drying stage was 8.5 hours at 4° C. The second stage was 6.5 hours at 25° C. Between the first and second stage, the temperature ramped from 4-25° C. over 4.5 hours. The pressure setpoint during the entire 21 hours was about 12 μBar. After drying, samples were backfilled with nitrogen gas, then stoppered and crimped.

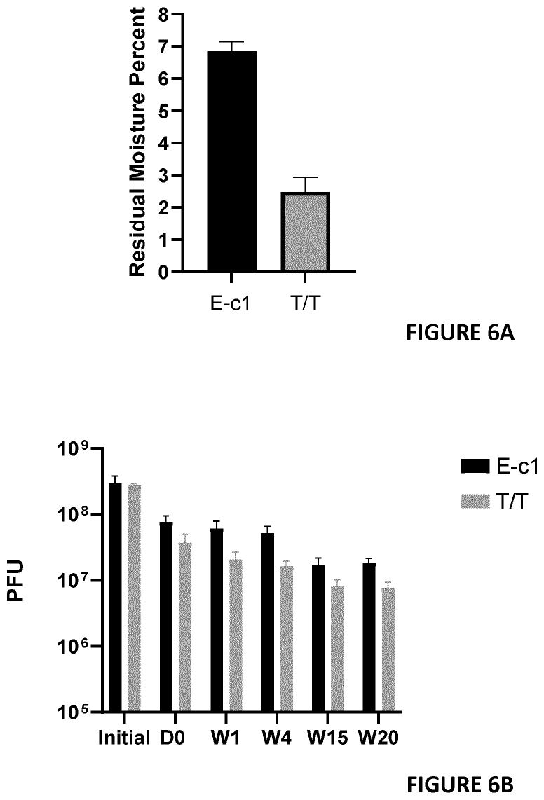

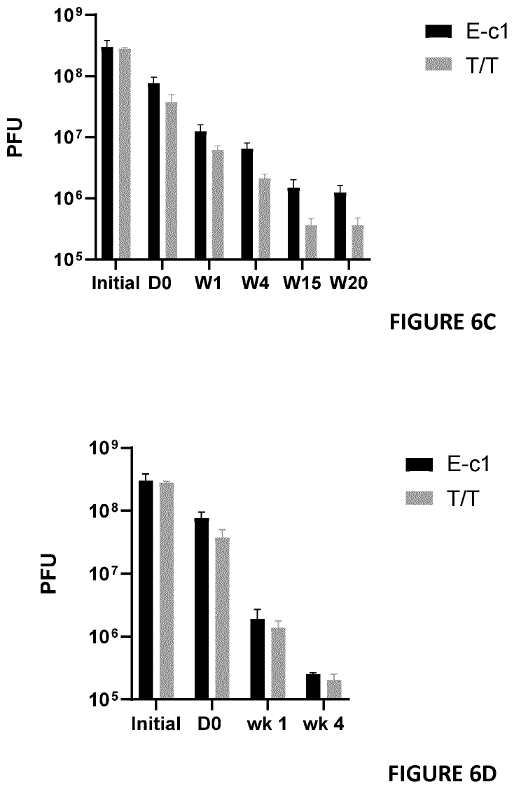

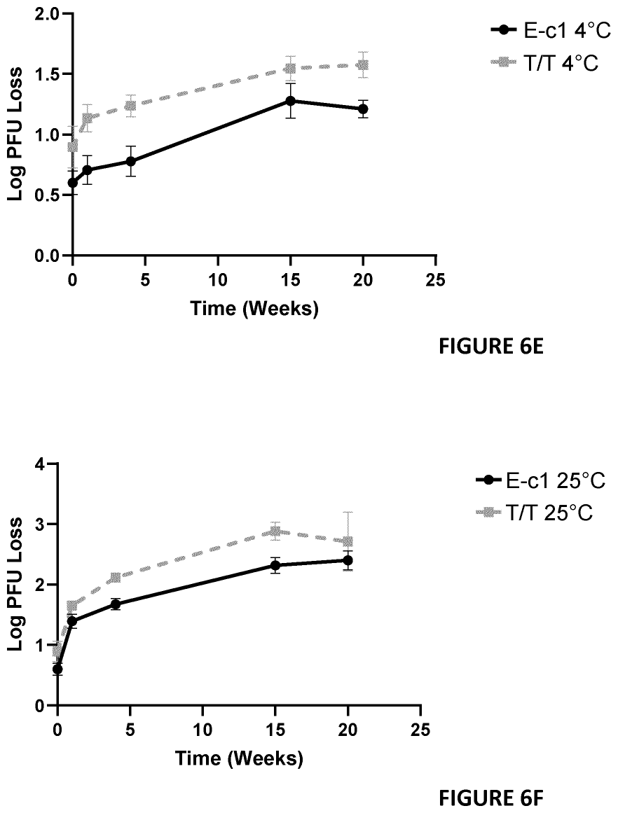

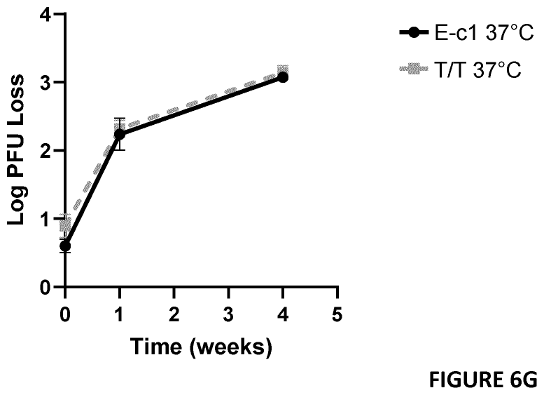

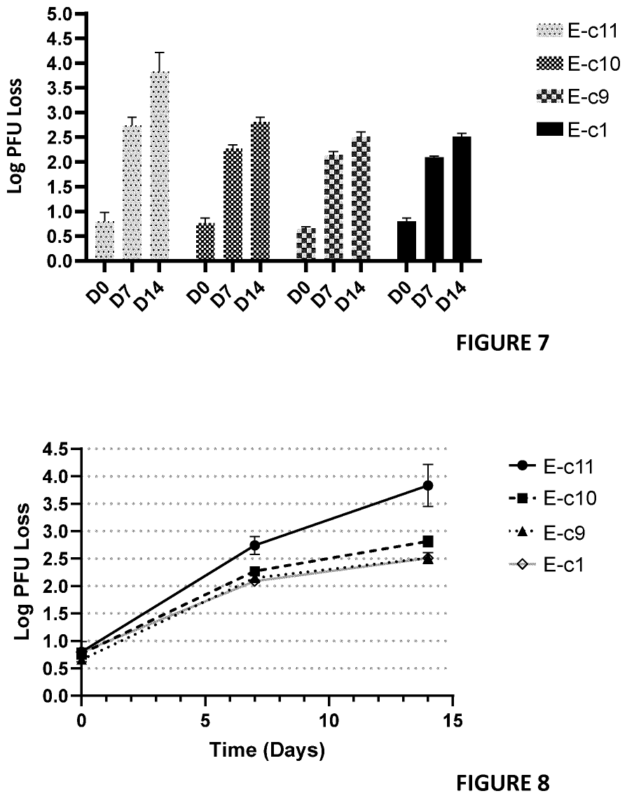

The residual moisture (RM) of the E-c1 formulation was about 6.8%. The residual moisture of the Trehalose formulation was about 2.5% (FIG. 6A). There was less than 1 log of process loss for both samples.

The samples were incubated at 4° C., 25° C. for 20 weeks and at 37° C. for 4 weeks. FIGS. 6B-6G present the results of this study.

### Example 17: PT/CM/BSA/NaCl Composition Dried at 4° C./25° C. With Varying PT Concentrations

Serum free VSV-GFP was transferred from an initial solution to a dialysis buffer solution by way of buffer exchange using a spin column to create two stock solutions.

Four formulations were prepared using 10% by volume of stock solution and 90% by volume of a solution of pullulan and trehalose, each dissolved in the dialysis buffers, with varying concentrations of pullulan and trehalose as indicated in Table 10 below.

TABLE 10
Formulations for The Study

| | E-c1 | E-c9 | E-c10 | E-c11 |
|---|---|---|---|---|
| VSV Stock | GFP (Lot 357) | | | |
| Dialysis Buffer (pH 7.2) | CM + 0.5% BSA + 50 mM NaCl | | | |
| Formulation (+Dialysis | 2.5% Pullulan | 1.25% Pullulan | 0.625% Pullulan | 0.25% Pullulan |
| Buffer) | 5% Trehalose | 2.5% Trehalose | 1.25% Trehalose | 0.625% Trehalose |

| | |
|---|---|
| Total Volume | 10 ul Virus + 90 ul formulation = 100 ul |

100 uL aliquots of the samples were foam dried in freeze dryer 2 mL glass vials for 23 hours using a two-stage drying protocol. The first (primary) drying stage was 10 hours at 4° C. The second stage was 6.5 hours at 25° C. Between the first and second stage, the temperature ramped from 4-25° C. over 6.5 hours. The pressure setpoint during the entire 23 hours was about 12 uBar. After drying, the samples were stoppered inside of dryer so only inert Nitrogen was in sample vial.

The residual moisture (RM) of the formulation showed minimum PT is 0.625% P, 1.25% T and RM reduced with higher PT concentrations (FIG. 8A). FIG. 8B shows the dry weight of the compositions.

The samples were incubated at 37° C. for two weeks. FIGS. 8C-8D present the results of this study. Process loss for all formulations was about 0.9 log. After 14 days, titer loss of about 2.5 log for 5% and 2.5% Trehalose formulations. Table 11 below shows the wt % of the components in the dry formulation.

TABLE 11

| | E-c1 | | E-c1 | E-c9 | | E-c9 | E-c10 | | E-c10 | E-c11 | | E-c11 |
|---|---|---|---|---|---|---|---|---|---|---|---|---|
| | (As made | | Wt % Dried | (As made | | Wt % Dried | (As made | | Wt % Dried | (As made | | (Wt % Dried |
| | Formulation) | | Formulation | Formulation) | | Formulation | Formulation) | | Formulation | Formulation) | | Formulation) |
| Pullulan | 2.5 | wt % | 28.5 | 1.25 | wt % | 24.9 | 0.625 | wt % | 19.8 | 0.25 | wt % | 11.5 |
| Trehalose | 5 | wt % | 57.0 | 2.5 | wt % | 49.7 | 1.25 | wt % | 39.7 | 0.625 | wt % | 29.0 |
| BSA | 0.5 | wt % | 6.3 | 0.5 | wt % | 11.1 | 0.5 | wt % | 17.6 | 0.5 | wt % | 25.8 |
| NaCl | 50 | mM | 3.7 | 50 | mM | 6.5 | 50 | mM | 10.3 | 50 | mM | 15.1 |
| Tris | 10 | mM | 1.5 | 10 | mM | 2.7 | 10 | mM | 4.3 | 10 | mM | 6.3 |
| Gelatin | 0.01 | wt % | 0.1 | 0.01 | wt % | 0.11 | 0.01 | wt % | 0.18 | 0.01 | wt % | 0.26 |
| MgSO4 | 10 | mM | 1.5 | 10 | mM | 2.7 | 10 | mM | 4.2 | 10 | mM | 6.2 |
| CaCl2 | 10 | mM | 1.4 | 10 | mM | 2.5 | 10 | mM | 3.9 | 10 | mM | 5.7 |

| | | | | | | | | |
|---|---|---|---|---|---|---|---|---|
| Virus | 1e8 | 0.001 | 1e8 | 0.0009 | 1e8 | 0.0014 | 1e8 | 0.0021 |
| | particles | | particles | | particles | | particles | |

### Example 18: PT/CM/BSA/NaCl Dialyzed and Non-Dialyzed Composition with and without Sucrose Dried at 15° C./25° C.

FBS free VSV-GFP was transferred from an initial solution to a dialysis buffer solution by way of buffer exchange using a Zebra spin column (0.5 mL columns) to create two stock solutions.

Four formulations were prepared using 10% by volume of stock solution and 90% by volume of a formulation solution, each dissolved in one of the dialysis buffers, as indicated in Table 12 below.

TABLE 12
Formulations for The Study

| | E-c1 | Sample 1 | Sample 2 | Sample 3 | Sample 4 |
|---|---|---|---|---|---|
| VSV Stock | GFP | Dialyzed | Non-Dialyzed | Dialyzed | Dialyzed |
| | (Lot 357) | 10 ul | 10 ul | 10 ul | 10 ul |
| Dialysis Buffer | CM + 0.5% | CM + 0.5% | [symbol] | CM + 0.5% | CM + 0.5% |
| (pH 7.2) | BSA + 50 mM | BSA + 50 mM | | BSA + 50 mM | BSA + 50 mM |
| | NaCl | NaCl | | NaCl | NaCl + 4% |
| | | | | | sucrose |
| Formulation | 2.5% Pullulan | 90 ul | 90 ul | 90 ul formulation | 90 ul |
| (+Dialysis Buffer) | 5% Trehalose | | | buffer + 490 ul | |
| | | | | dialysis buffer | |
| Total Volume | 100 | 100 ul | 100 ul | 590 ul | 100 ul |

The samples were dried in freeze dryer in 2 mL glass vials for 23 hours using a two-stage drying protocol. The first (primary) drying stage was 10 hours at 15° C. The second stage was 6.5 hours at 25° C. Between the first and second stage, the temperature ramped from 15-25° C. over 6.5 hours. The samples were pre-cooled at 15° C. for 30 minutes before starting the drying. The pressure setpoint during the entire 21 hours was about 12 μBar. After drying, samples were backfilled with nitrogen gas, then stoppered and crimped.

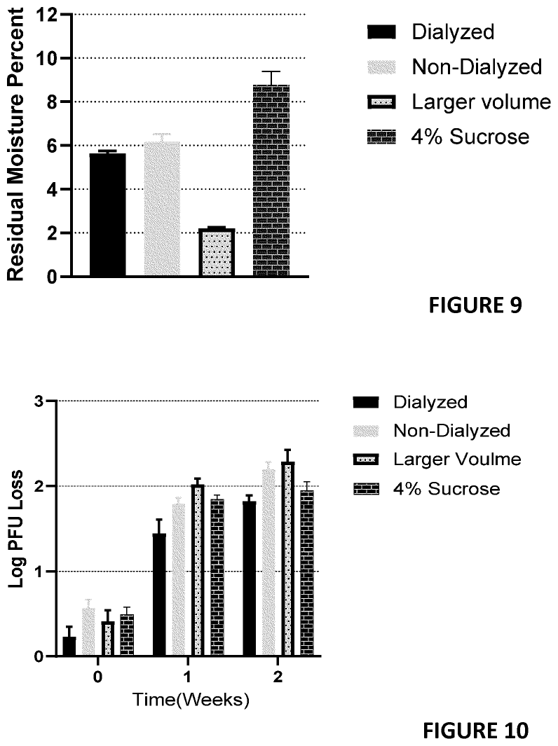

FIG. 9 presents the RM after drying of the samples.

The samples were incubated at 37° C. for 14 days. FIG. 10 present the results of this study.

### Example 19: PT Dried Under Ambient Pressure and Temperature

Serum free VSV was obtained in a stock solution containing 100 mM HEPES buffer, 150 mM NaCl and 4% sucrose. Aliquots were prepared by mixing 1 uL of a serum free VSV stock solution with 9 uL of a solution containing 10 wt % pullulan and 20 wt % trehalose in water in 1.7 mL centrifuge tubes. Residual moisture of the samples was not measured since the samples were too small for accurate measurement. The initial (before drying) titer of each aliquot was approximately 10^{7.6} PFU. The aliquots were air dried in a biosafety cabinet for 3 days at ambient pressure and 25 C. After drying, the aliquots were below the detection limit (10³ PFU) of the titer assay.

### Example 20: CM/BSA/NaCl/PT Dried Under Ambient Pressure and Temperature

Serum free VSV was transferred from a stock solution to a CM buffer. Aliquots were prepared by mixing 1 uL of the VSV/CM buffer solution into 9 uL of a solution having 0.5% BSA, 50 mM NaCl, 2.5 wt % pullulan and 5 wt % trehalose in CM buffer in 1.7 mL centrifuge tubes (E-c1). The initial (before drying) titer of each aliquot was approximately 10^{7.6} PFU. The aliquots were air dried in a biosafety cabinet for 3 days at ambient pressure and 25° C.

Residual moisture of the samples was not measured since the samples were too small for accurate measurement. Aliquots tested immediately after drying (DO) had a titer of approximately 10^{5.8} PFU, indicating a process loss of about 1.8 logs. Aliquots tested after drying and incubation at 37° C. for 7 days (D7) had a titer of approximately 10^{4.5} PFU, indicating a total loss of over 3 logs.

### Example 21 (CM/BSA/NaCl/PT 800 mBar Vacuum Dried)

Serum free VSV was transferred from an initial solution to a dialysis buffer solution by way of buffer exchange using a spin column. A mixture was prepared having 10% by volume of the VSV in CM buffer and 90% by volume of a solution having 0.5% BSA, 50 mM NaCl, 2.5 wt % pullulan and 5 wt % trehalose in CM buffer. 100 uL aliquots of the mixture were transferred to 2 mL glass vials. The initial titer of the samples was 10^{8.2} PFU. The aliquots were dried at 4 C for 22 hours at a pressure of 800 mBar. At the end of the 22 hours, the samples were still liquid. The samples were then dried for a further 22 hours at 800 mBar with the temperature ramping from 4 C to 25C for 9 hours and then held at 25 C for 13 hours. After the entire 44 hours of drying, a clear film had formed in the vials and the vials were stoppered and crimped. The residual moisture was 10%. Samples tested immediately after drying had a titer of 106.7 PFU indicating a process loss after drying of 1.5 log.

Aliquots of another mixture prepared as described in the paragraph above were dried at 8 C for 45 hours at a pressure of 800 mBar. The initial titer of the samples was 10^{8.3} PFU. After the 45 hours of drying, a clear film had formed in the vials and the vials were stoppered and crimped. The residual moisture in was 8%. Samples tested immediately after drying had a titer of 10⁷ PFU indicating a process loss after drying of 1.3 log.

No foam was formed in these experiments. The process loss with both vacuum drying methods was higher than for any foam drying method tested. Heat challenge tests on the vacuum dried samples were not conducted.

### Example 22: Various Formulations Dried at 4° C./25° C.

Serum free VSV was transferred from an initial solution to a dialysis buffer solution by way of buffer exchange using a spin column to create a stock solution. The dialysis buffer contained 10 mM Tris-HCl.

Five formulations were prepared using 10% by volume of the stock solution and 90% by volume of a solution as indicated in Table 1. 100 uL aliquots of the samples were foam dried in freeze dryer in 2 mL glass vials. The drying temperature was 4° C. for 10 hours, followed by 6.5 hours ramping from 4° C. to 25° C., followed by 6.5 hours at 25° C. The cabinet pressure set point during the 23 hours drying cycle was 17 uBar. After drying, samples were backfilled with nitrogen gas, then stoppered and crimped.

TABLE 13
Formulations for The Study

| | | Tris PT | Tris PT | Tris PT | |
|---|---|---|---|---|---|
| | Tris PT | NaCl | BSA | NaCl BSA | E-c1 |
| Tris | 10 mM | 10 mM | 10 mM | 10 mM | 10 mM |
| MgSO4 | | | | | 10 mM |
| CaCl2 | | | | | 10 mM |
| Gelatin | | | | | 0.05% |
| BSA | | | 0.5% | 0.5% | 0.5% |
| NaCl | | 50 mM | | 50 mM | 50 mM |
| Pullulan | 2.5% | 2.5% | 2.5% | 2.5% | 2.5% |
| Trehalose | 5% | 5% | 5% | 20% | 5% |

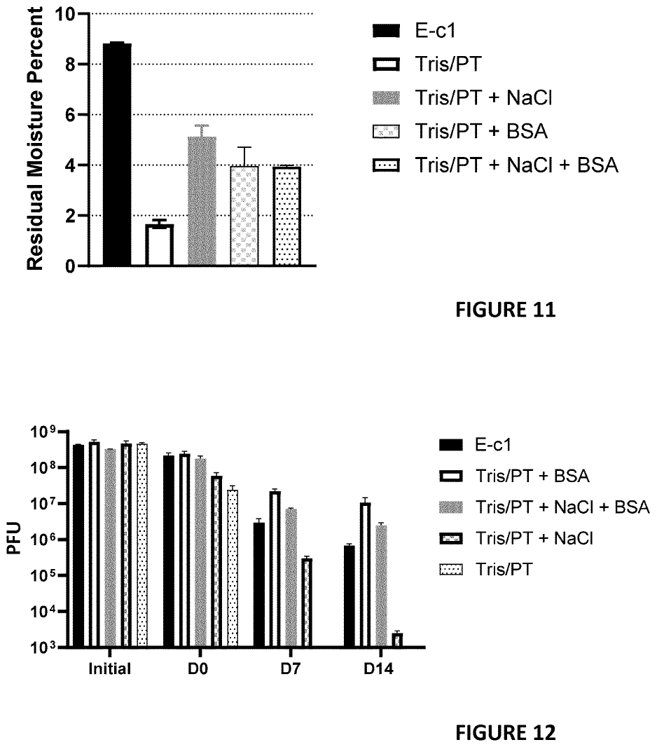

Residual moisture of the samples after drying is shown in FIG. 11.

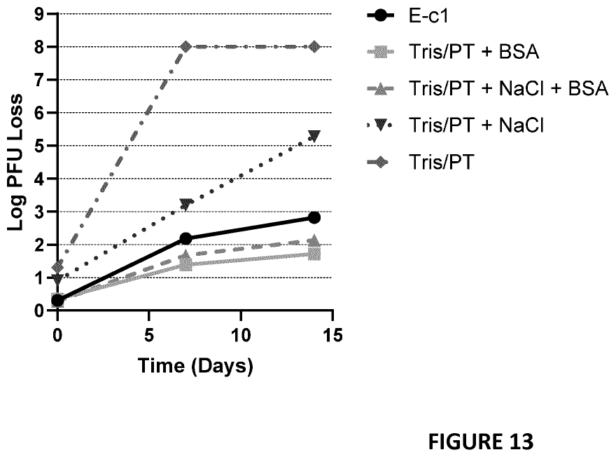

Samples were tested before drying (Initial), immediately after drying (D0), after 7 days of incubation at 37° C. (D7) and after 14 days of incubation at 37° C. (D14). Process loss was about 0.3 log for BSA containing formulations. Tris PT had a process loss of 1.3 log and PFU counts below the detection limits of the assay at D7 and D14. Other data is presented in FIGS. 12 and 13. The data for Tris PT was considered unreliable and re-tested in the example below.

TABLE 14
Wt % of Formulations

| | | | | Tris/PT + | | Tris/PT + | Tris/PT + | | Tris/PT + | Tris/PT + | | Tris/PT + | | | |
|---|---|---|---|---|---|---|---|---|---|---|---|---|---|---|---|
| | E-c1 | | E-c1 | BSA | | BSA | NaCl + BSA | | NaCl + BSA | NaCl | | NaCl | Tris/PT | | Tris/PT |
| | (As made | | Wt % Dried | (As made | | Wt % Dried | (As made | | (Wt % Dried | (As made | | (Wt % Dried | (As made | | (Wt % Dried |
| | Formul- | | Formu- | Formu- | | Formu- | Formu- | | Formu- | Formu- | | Formu- | Formu- | | Formu- |
| | ation) | | lation | lation) | | lation | lation) | | lation) | lation) | | lation) | lation) | | lation) |
| Pullulan | 2.5 | wt % | 28.5 | 2.5 | wt % | 30.5 | 2.5 | wt % | 29.4 | 2.5 | wt % | 31.4 | 2.5 | wt % | 32.7 |
| Trehalose | 5 | wt % | 57.0 | 5 | wt % | 61.0 | 5 | wt % | 58.7 | 5 | wt % | 62.8 | 5 | wt % | 65.5 |
| BSA | 0.5 | wt % | 6.3 | 0.5 | wt % | 6.8 | 0.5 | wt % | 6.5 | — | | — | — | | — |
| NaCl | 50 | mM | 3.7 | — | | — | 50 | mM | 3.8 | 50 | mM | 4.1 | — | | 0.0 |
| Tris | 10 | mM | 1.5 | 10 | mM | 1.6 | 10 | mM | 1.6 | 10 | mM | 1.7 | 10 | mM | 1.8 |

| | | | | | | | | | | | |
|---|---|---|---|---|---|---|---|---|---|---|---|
| Gelatin | 0.01 | wt % | 0.1 | — | — | — | — | — | — | — | — |
| MgSO4 | 10 | mM | 1.5 | — | — | — | — | — | — | — | — |
| CaCl2 | 10 | mM | 1.4 | — | — | — | — | — | — | — | — |

| | | | | | | | | | | |
|---|---|---|---|---|---|---|---|---|---|---|
| Virus | 1e8 | 0.001 | 1e8 | 0.0005 | 1e8 | 0.0005 | 1e8 | 0.0006 | 1e8 | 0.0006 |
| | particles | | particles | | particles | | particles | | particles | |

### Example 23: Various Formulations Dried at 4° C./25° C.

Serum free VSV was transferred from an initial solution to a dialysis buffer solution by way of buffer exchange using a spin column to create a stock solution. The dialysis buffer contained 10 mM Tris-HCl.

Formulations were prepared using 10% by volume of the stock solution and 90% by volume of a solution as indicated in Table 1. 100 uL aliquots of the samples were foam dried in freeze dryer in 2 mL glass vials. The drying temperature was 4° C. for 10 hours, followed by 6.5 hours ramping from 4° C. to 25° C., followed by 6.5 hours at 25° C. The cabinet pressure set point during the 23 hours drying cycle was 17 uBar. After drying, samples were backfilled with nitrogen gas, then stoppered and crimped.

TABLE 15
Formulations for The Study

| | | | | | | | | | | Tris/ | | Tris/ | | | Tris/ |
|---|---|---|---|---|---|---|---|---|---|---|---|---|---|---|---|
| | | | A | | | Tris/PT | Tris/PT + | | Tris/PT + | Trehalose + | | Trehalose + | Tris/ | | Trehalose |
| | A | | Wt % | Tris/PT | | (Wt % | BSA | | BSA Wt % | BSA | | BSA (Wt % | Trehalose | | (Wt % |
| | (As made | | Dried | (As made | | Dried | (As made | | Dried | (As made | | Dried | (As made | | Dried |
| | Formu- | | Formu- | Formu- | | Formu- | Formu- | | Formu- | Formu- | | Formu- | Formu- | | Formu- |
| | lation) | | lation | lation) | | lation) | lation) | | lation | lation) | | lation) | lation) | | lation) |
| Pullulan | 2.5 | wt % | 28.5 | 2.5 | wt % | 32.7 | 2.5 | wt % | 30.5 | — | | — | — | | — |
| Trehalose | 5 | wt % | 57.0 | 5 | wt % | 65.5 | 5 | wt % | 61.0 | 5 | wt % | 87.9 | 5 | wt % | 97.4 |
| BSA | 0.5 | wt % | 6.3 | — | | — | 0.5 | wt % | 6.8 | 0.5 | wt % | 9.8 | — | | — |
| NaCl | 50 | mM | 3.7 | — | | — | — | | — | — | | — | — | | — |
| Tris | 10 | mM | 1.5 | 10 | mM | 1.8 | 10 | mM | 1.6 | 10 | mM | 2.4 | 10 | mM | 2.6 |

| | | | | | | | | | | | |
|---|---|---|---|---|---|---|---|---|---|---|---|
| Gelatin | 0.01 | wt % | 0.1 | — | — | — | — | — | — | — | — |
| MgSO4 | 10 | mM | 1.5 | — | — | — | — | — | — | — | — |
| CaCl2 | 10 | mM | 1.4 | — | — | — | — | — | — | — | — |

| | | | | | | | | | | |
|---|---|---|---|---|---|---|---|---|---|---|
| Virus | 1e8 | 0.001 | 1e8 | 0.0006 | 1e8 | 0.0005 | 1e8 | 0.001 | 1e8 | 0.001 |
| | particles | | particles | | particles | | particles | | particles | |

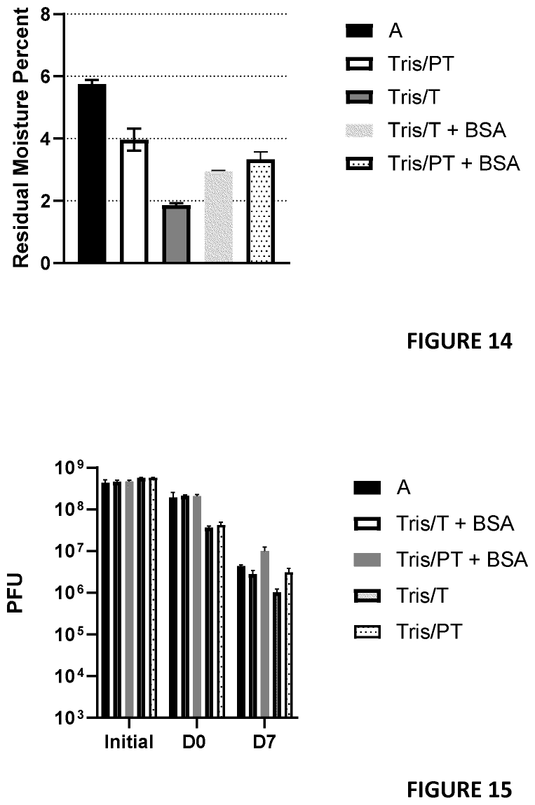

Residual moisture of the samples after drying is shown in FIG. 14.

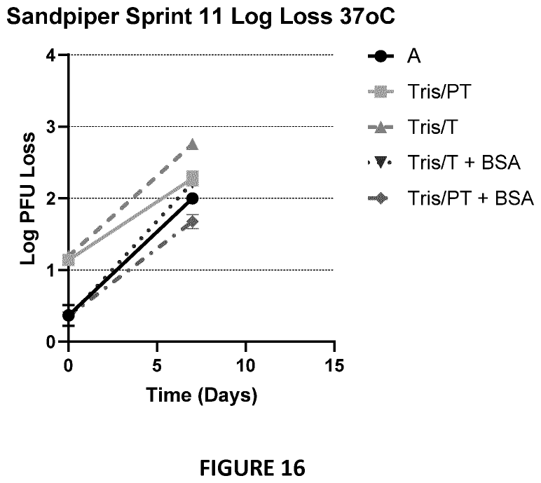

Samples were tested before drying (Initial), immediately after drying (D0), after 7 days of incubation at 37° C. (D7) and after 14 days of incubation at 37° C. (D14). Data is presented in FIGS. 15 and 16.

### Example 24

AdV-GFP was transferred from its stock buffer into a sample buffer by buffer exchange using 0.5 mL Zeba columns. 10 parts by volume of the sample buffer was mixed with 90 parts by volume of a solution of additional excipients dissolved in an additional amount of the sample buffer.

In Formulation A, the sample buffer has 10 mM Tris-HCl (pH 7.2), 10 mM MgSO₄, 10 mM CaCl₂, 0.005% Gelatin, 0.5% BSA and 50 mM NaCl. The additional excipients are 2.5% Pullulan and 5% Trehalose. Formulation A on a fully dried basis has the following components: Pullulan 28.5 wt %; Trehalose 57.0 wt %; BSA 6.3 wt %; NaCl 3.7 wt %, Tris 1.5 wt %; Gelatin 0.1 wt %; MgSO₄ 1.5 wt %; CaCl₂1.4 wt %; and, virus 0.001 wt %.

In Formulation B (Inulin/Mannitol), the sample buffer has 10 mM Tris-HCl (pH 8.2), 1 mM MgSO₄ and 100 mM NaCl. The additional excipients are 5% Inulin and 5% Mannitol. Formulation B represents a formulation described in Berg et al. 2021 for freeze drying Chimp AdV. This publication reports an infectivity loss of about 2 log after 30 days of storage at 45 C, and an infectivity loss of about 1.5 log after 60 days of storage at 30 C.

The samples were foam dried. 100 uL samples of each formulation were placed in vials and pre-cooled to 4C. The pre-cooled vials were transferred to a lab scale freeze dryer operated as foam dryer. The pressure setpoint was 16 uBar pressure. The temperature schedule was 10 hours at 4 C, 6.5 hours of a gradient from 4 C to 25 C, and 6.5 hours at 25 C.

After foam drying, the residual moisture (RM) of Formulation A was 6.0%. The RM of Formulation B was 2.3%.

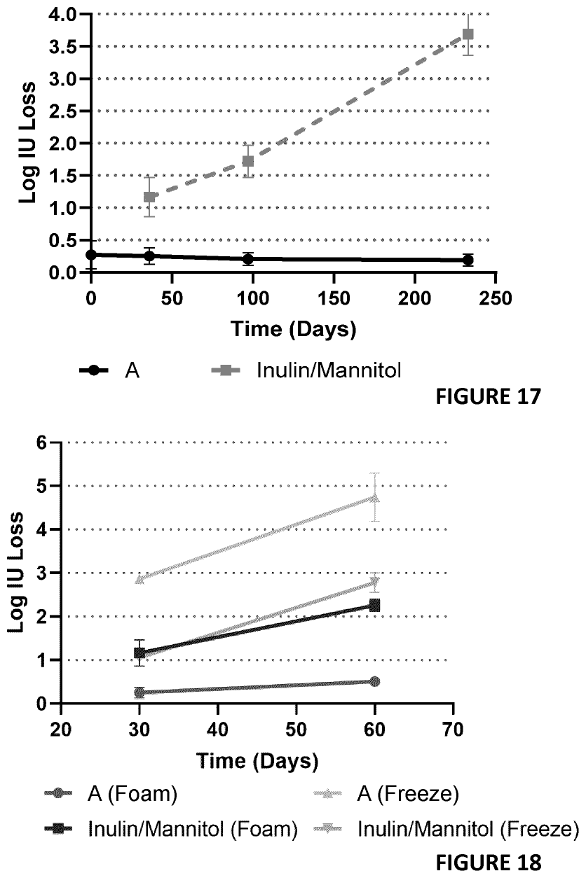

Before drying, each sample had about 10⁸ infectious units (IU). The dried samples were stored at 37 C and tested for IU using an in-cell Western (ICW) assay with HEK293 cells. IU loss relative to the initial (before buffer exchange) IU of each formulation after drying (DO) and after storage for various time periods is shown in FIG. 17.

### Example 25

Addition samples were prepared according to Formulations A and B and foam dried as described above.

Another group of samples according to Formulations A and B were freeze dried over a 63 hour drying cycle. The primary stages of the cycle were: freezing under atmospheric pressure at −50 C; primary drying at −50 C (all temperatures are shelf temperature) and 0.03 mbar; secondary drying at 20 C and 0.03 mbar; and, tertiary drying at 20 C and 750 mbar. After After freeze drying, the residual moisture (RM) of Formulation A was 0.8%. The RM of Formulation B was 0.4%.

Before drying, each sample had about 10⁸ infectious units (IU). The dried samples were stored at 37 C and tested for IU using an in-cell Western (ICW) assay with HEK293 cells. IU loss measured after storage relative to the initial IU of each formulation is shown in FIG. 18.

### Example 26

Additional samples of Formulation A and Formulation B were prepared. A third formulation, Formulation C, was also prepared as described above. In Formulation C (Tris/5% Trehalose), the sample buffer has 10 mM Tris-HCl (pH7.2). The additional excipients were 5% Trehalose.

100 uL samples of each formulation were placed in vials and foam dried. The samples were pre-cooled to 4C. The pre-cooled vials were transferred to a lab scale freeze dryer operated as foam dryer. The pressure setpoint was 16 uBar pressure. The temperature schedule was 10 hours at 4 C, 6.5 hours of a gradient from 4 C to 25 C, and 6.5 hours at 25 C. The dryer was backfilled with nitrogen gas and the vials were stoppered inside the dryer, and crimpled immediately after being removed from the dryer.

The residual moisture (RM) of Formulation A was 7.0%. The RM of Formulation B was 1.9%. The RM of formulation C was 1.1%.

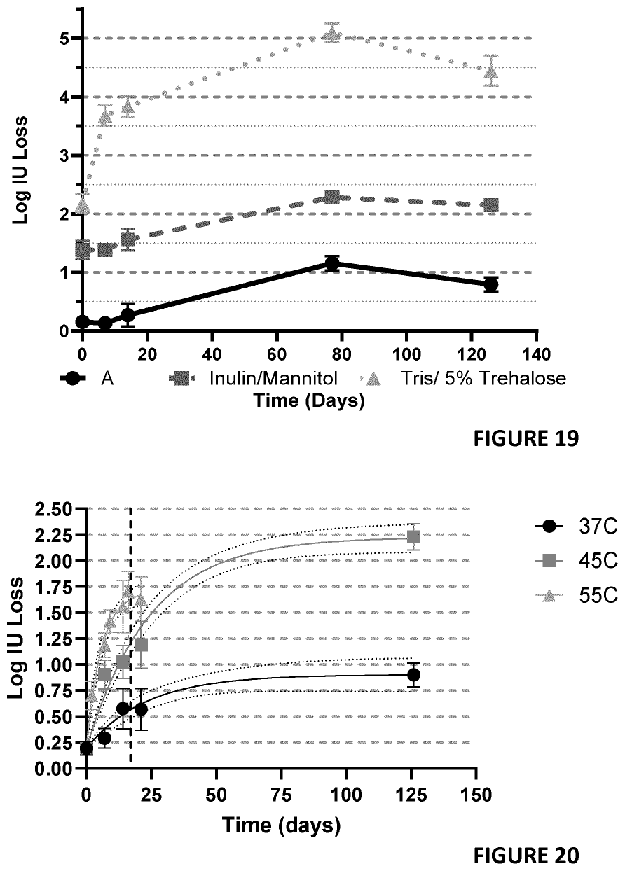

Before drying, each sample had about 10⁸ infectious units (IU). The dried samples were stored at 37 C and tested for IU using an in-cell Western (ICW) assay with HEK293 cells. IU loss relative to the initial (before buffer exchange) IU of each formulation after drying (DO) and after storage for various time periods is shown in FIG. 19.

### Example 27

Additional samples of Formulation A were prepared.

100 uL samples of each formulation were placed in vials and foam dried. The samples were pre-cooled to 4C. The pre-cooled vials were transferred to a lab scale freeze dryer operated as foam dryer. The pressure setpoint, initiated immediately after the pre-cooling period, was 11 uBar pressure. The temperature schedule was 10 hours at 4 C, 6.5 hours of a gradient from 4 C to 25 C, and 6.5 hours at 25 C. The dryer was backfilled with nitrogen gas and the vials were stoppered inside the dryer, and crimpled immediately after being removed from the dryer.

Unless stated otherwise, temperatures described herein are temperature setpoints which are controlled based on the temperature of the shelf inlet of the dryer. Probes placed on the wall of vials indicate that the temperature of the vials drops to below-10C for several minutes after the pressure setpoint is implemented and remains below OC for roughly 50-100 minutes.

The residual moisture (RM) of Formulation A was 6.8%.

Before drying, each sample had about 10⁸ infectious units (IU). The dried samples were stored at 37 C, 45 C or 55 C and tested for IU using an in-cell Western (ICW) assay with HEK293 cells. IU loss relative to the initial (before buffer exchange) IU of Formulation A after drying (DO) and after storage for various time periods is shown in FIG. 20.

### Example 28

Additional samples of Formulation A were prepared. A fourth formulation, Formulation D (Tris PT), was also prepared as described above. In Formulation D, the sample buffer has 10 mM Tris-HCl (pH7.2). The additional excipients were 2.5% pullulan and 5% Trehalose. A fifth formulation, Formulation E, was also prepared as described above. In Formulation E, the sample buffer has 10 mM Tris-HCl (pH 7.2), 10 mM MgSO₄, 10 mM CaCl2, 0.005% Gelatin, 0.5% BSA and 50 mM NaCl. The additional excipients are 2.5% Pullulan and 5% Trehalose and 2% Sorbitol.

100 uL samples of each formulation were placed in vials and foam dried. The samples were pre-cooled to 4C. The pre-cooled vials were transferred to a lab scale freeze dryer operated as foam dryer. The pressure setpoint was 12 uBar pressure. The temperature schedule was 10 hours at 4 C, 6.5 hours of a gradient from 4 C to 25 C, and 6.5 hours at 25 C. The dryer was backfilled with nitrogen gas and the vials were stoppered inside the dryer, and crimpled immediately after being removed from the dryer.

The residual moisture (RM) of Formulation A was 6.8%. The RM of Formulation D was 4.4%. The RM of formulation E was 12.2%.

Before drying, each sample had about 10⁸ infectious units (IU). The dried samples were stored at 55 C and tested for IU using an in-cell Western (ICW) assay with HEK293 cells. IU loss relative to the initial (before buffer exchange) IU of each formulation after after storage for 14 days was, Formulation A: 1.8 log loss; Formulation D: 1.2 log loss; and, Formulation E: 1.6 log loss.

### Example 29

Additional samples of Formulation A and D were prepared.

100 uL samples of each formulation were placed in vials and foam dried. The vials were transferred to a lab scale freeze dryer operated as foam dryer. The pressure setpoint was 15 uBar pressure. The temperature schedule was 23 hours at 25 C. The dryer was backfilled with nitrogen gas and the vials were stoppered inside the dryer, and crimpled immediately after being removed from the dryer. Despite the higher setpoint temperature, the temperature as indicated by probes attached the vials still dropped below OC for a period of time.

The residual moisture (RM) of Formulation A was 5%. The RM of Formulation D was 4%.

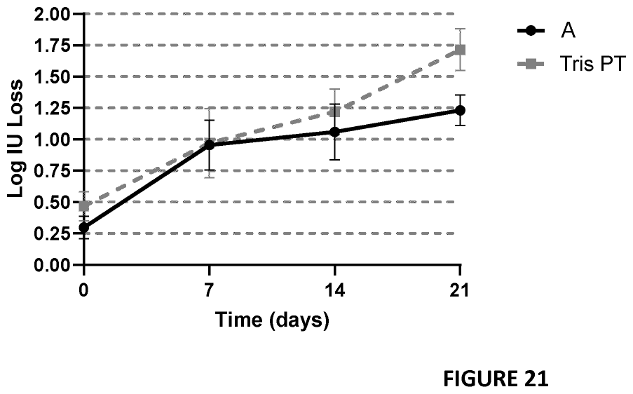

Before drying, each sample had about 10⁸ infectious units (IU). The dried samples were stored at 55 C and tested for IU using an in-cell Western (ICW) assay with HEK293 cells. IU loss relative to the initial (before buffer exchange) IU of each formulation after storage is shown in FIG. 21.

### Example 30

Additional samples of Formulation A were prepared. A sixth formulation, Formulation F, was also prepared as described above. In Formulation F, the sample buffer has 10 mM Tris-HCl (pH7.2). The additional excipients were 2.5% pullulan, 5% Trehalose and 0.5% BSA.

100 uL samples of each formulation were placed in vials and foam dried. The vials were transferred to a lab scale freeze dryer operated as foam dryer. The pressure setpoint was 15 uBar pressure. The temperature schedule was 23 hours at 25 C. The dryer was backfilled with nitrogen gas and the vials were stoppered inside the dryer, and crimpled immediately after being removed from the dryer. Despite the higher setpoint temperature, the temperature as indicated by probes attached the vials still dropped below OC for a period of time.

The residual moisture (RM) of Formulation A was about 4.6%. The RM of Formulation F was about 3.3%.

Before drying, each sample had about 10⁸ infectious units (IU). The dried samples were stored at 55 C and tested for IU using an in-cell Western (ICW) assay with HEK293 cells. IU loss relative to the initial (before buffer exchange) IU of each formulation after storage for 7 days was about 0.8 log for formulation A and about 0.9 log for Formulation F.

1. A method of preserving and/or stabilizing a virus, the method comprising:
combining the virus with trehalose, water and a buffer, optionally with pullulan and/or albumin to produce a liquid composition; and,
drying the liquid composition to produce a dried composition,
wherein drying the liquid composition comprises exposing the liquid composition to a vacuum sufficient to produce bubbles in the liquid composition while removing water from the composition to produce an intermediate composition, and reducing the residual moisture of the intermediate composition to produce the dried composition.

2. The method of claim 1 wherein the virus is a live-attenuated virus and/or a viral vector.

3. The method of claim 1, wherein the virus is derived from a vesicular stomatitis virus (VSV).

4. The method of claim 1, wherein the virus is derived from an adenovirus (AdV).

5. The method of claim 1 wherein the dried composition comprises a foamed glass encapsulating the virus.

6. The method of claim 1 wherein the liquid composition is maintained at a temperature in the range of 1 C to 15 C, or in the range of 1 C to 10 C, before exposing the liquid composition to the vacuum.

7. The method of claim 1 wherein reducing the residual moisture of the intermediate composition comprises exposing the intermediate composition to a vacuum while heating the intermediate composition, for example to a temperature in the range of 20 C to 40 C.

8. The method of claim 1 wherein the vacuum sufficient to produce bubbles in the liquid composition has a pressure in the range of 5 μBar to 100 mBar 5 μBar to 50 mBar, 5 μBar to 10 mBar, 5 μBar to 5 mBar, 5-200 μBar or 5-20 μBar.

9. The method of claim 1 wherein the composition comprises albumin.

10. The method of claim 1 wherein the composition comprises pullulan.

11-13. (canceled)

14. The method of claim 1 wherein the drying is carried out at two or more temperatures.

15. The method of claim 1 wherein the mixture is dried to a moisture content of about 1% to about 10%.

16. The method of claim 1 wherein the composition is a vaccine, a part of a vaccine or a precursor of a vaccine.

17. (canceled)

18. A composition comprising,
a virus;
trehalose;
less than 10% water,
a buffer; and,
optionally, pullulan and/or albumin

19. The method of claim 18 wherein the virus is a live-attenuated virus and/or a viral vector derived from a vesicular stomatitis virus (VSV) or derived from an adenovirus (AdV).

20. (canceled)

21. (canceled)

22. The method of claim 18 wherein the composition comprises a foamed glass encapsulating the virus.

23. (canceled)

24. (canceled)

25. The composition of claim 18 comprising less than 1 wt % on a fully dried basis of sulphate and phosphate salts.

26-28. (canceled)

29. The composition of claim 18 comprising about 23 to about 300 parts of the trehalose, about 1 to about 10 parts of the buffer, about 12 to about 150 parts of the pulllulan and about 2 to about 35 parts of the albumin, and about 0.0001 to about 0.01 parts of the virus, by weight.

30. The composition of claim 18 wherein the composition is a vaccine, a part of a vaccine or a precursor of a vaccine.

31-60. (canceled)
